(19) United States
(12) Patent Application Publication
(10) Pub. No.: US 2026/0008824 A1
(43) Pub. Date: Jan. 8, 2026

(54) PLANT-PRODUCED mABs AGAINST CHIKUNGUNYA VIRUS WITH ENHANCED EFFECTOR FUNCTION AND EFFICACY

(71) Applicant: Arizona Board of Regents on Behalf of Arizona State University, Tempe, AZ (US)

(72) Inventors: Qiang Chen, Chandler, AZ (US); Jonathan Hurtado, Chandler, AZ (US); Haiyan Sun, Chandler, AZ (US); Huafang Lai, Chandler, AZ (US)

(21) Appl. No.: 19/278,230

(22) Filed: Jul. 23, 2025

Related U.S. Application Data
(63) Continuation of application No. 17/615,803, filed on Dec. 1, 2021, now Pat. No. 12,466,862.
(60) Provisional application No. 62/857,081, filed on Jun. 4, 2019.

(51) Int. Cl. C07K 14/415; C07K 16/10; C12N 15/82

(52) U.S. Cl. CPC C07K 14/415; C07K 16/1081; C12N 15/8258; C07K 2317/13; C07K 2317/41; C07K 2317/732; C07K 2317/76

(57) ABSTRACT

Disclosed are monoclonal antibodies against Chikungunya virus having enhanced effector function and efficacy, methods of producing them, and methods of treatment of Chikungunya viral infection using the antibodies.

### CROSS-REFERENCE TO RELATED APPLICATIONS

This application is a continuation of U.S. 371 National patent application Ser. No. 17/615,803 filed Dec. 1, 2021, which claims the benefit from International Application No. PCT/US2020/036136 filed Jun. 4, 2020, which claims the benefit of U.S. Provisional Patent Application No. 62/857,081 filed Jun. 4, 2019, the entire disclosure of which is herein incorporated by reference.

### SEQUENCE LISTING

The present application contains a Sequence Listing which has been submitted electronically in .XML format and is hereby incorporated herein by reference in its entirety. Said computer readable file was created on Jul. 21, 2025, named 055743-853081_(M19-282L)_Sequence Listing.xml and is 49,152 bytes in size.

### FIELD OF THE INVENTION

The present disclosure generally relates to monoclonal antibodies against Chikungunya virus having enhanced effector function and efficacy.

### BACKGROUND OF THE INVENTION

Infection of CHIKV in humans can cause debilitating polyarthralgia that may persist months to years and affects multiple joints including ankles, knees, wrists, and fingers. Some of the symptoms are caused by the self-damaging host immune responses to CHIKV infection. Since 2004, CHIKV reemergence has resulted in millions of cases of severe and often chronic arthralgia on five continents. Currently, no licensed therapeutics or vaccines are available for human use against CHIKV. Continued outbreaks of CHIKV and the risk of it spreading into new areas call for the urgent development of effective preventatives and therapeutics.

Increasingly, greater attention is being focused on the production and use of larger and more complex protein molecules as therapeutic agents. Examples of such therapeutic proteins include antigens used in vaccinations to induce immune responses and antibodies. The potential utility of antibodies as efficacious anti-CHIKV therapeutics was suggested by early vaccine studies showing that vaccine potency correlates with its ability of inducing neutralizing antibodies in mice. Neutralizing antibodies from past infections in humans also shows evidence of protectivity, as these subjects are immune to CHIKV reinfection. Monoclonal antibodies (mAbs) against CHIKV E1 and E2 have been shown to be protective against CHIKV infection in various mouse models.

A need remains however for mAbs against CHIKV with enhanced efficacy in humans, and for improved methods of making such mAbs.

### SUMMARY OF THE INVENTION

One aspect of the present disclosure encompasses a monoclonal antibody having specificity to Chikungunya virus (CHIKV) E1 or E2 protein. The antibody is a glycosylated IgG antibody comprising a targeted, defined N-linked glycan profile of the Fc domain of at least one of the heavy chains with a high degree of glycan uniformity (homogeneity). The glycan profile can be an N-linked GlcNAc2Man3GlcNAc2 (GnGn) core glycan, (Man=Mannose, GlcNAc=N-acetylglucosamine), and the glycosylation profile can be more than about 95% uniform.

The antibody can be produced in a plant, plant fragment or plant cell. The plant can be ΔXFT N. benthamiana.

The antibody can be a human antibody. The antibody can comprise a heavy chain comprising an amino acid sequence at least 90% identical to SEQ. ID NO: 3; and a light chain comprising an amino acid sequence at least 90% identical to SEQ. ID NO: 5. The antibody can also comprise a heavy chain variable domain encoded by an amino acid sequence with at least about 90% identity to SEQ ID NO: 11; and a light chain variable domain encoded by an amino acid sequence with at least about 90% identity to SEQ ID NO: 13.

The antibody can comprise a heavy chain comprising an amino acid sequence at least 90% identical to SEQ. ID NO: 7; and a light chain comprising an amino acid sequence at least 90% identical to SEQ. ID NO: 9 . . . . The antibody can also comprise a heavy chain variable domain encoded by an amino acid sequence with at least about 90% identity to SEQ ID NO: 15; and a light chain variable domain encoded by an amino acid sequence with at least about 90% identity to SEQ ID NO: 17.

In some aspects, the antibody does not contribute to antibody-dependent enhancement (ADE) of virus infection. In an alternative of the aspects, the antibody does not contribute to antibody-dependent enhancement (ADE) of dengue virus infection.

Another aspect of the present disclosure encompasses a plant, plant fragment, or plant cell comprising a monoclonal antibody having specificity to CHIKV E1 or E2 protein, wherein the antibody is a glycosylated IgG antibody comprising a targeted, defined N-linked glycan profile of the Fc domain of at least one of the heavy chains with a high degree of glycan uniformity (homogeneity). The glycosylation profile can be an N-linked GlcNAc2Man3GlcNAc2 (GnGn) core glycan. (Man=Mannose, GlcNAc=N-acetylglucosamine). The plant can be ΔXFT N. benthamiana. In some aspects, the antibody does not contribute to antibody-dependent enhancement (ADE) of virus infection. In an alternative of the aspects, the antibody does not contribute to antibody-dependent enhancement (ADE) of dengue virus infection.

Yet another aspect of the present disclosure encompasses a composition for the prevention or treatment of CHIKV. The composition comprises a prophylactically or therapeutically effective amount of a monoclonal antibody having specificity to CHIKV E1 or E2 protein. The antibody is a glycosylated IgG antibody comprising a targeted, defined N-linked glycan profile of the Fc domain of at least one of the heavy chains with a high degree of glycan uniformity (homogeneity).

One aspect of the present disclosure encompasses a monoclonal antibody having specificity to CHIKV E1 protein. The antibody comprises a heavy chain variable domain encoded by an amino acid sequence with at least about 90% identity to SEQ ID NO: 11; and a light chain variable domain comprising an amino acid sequence at least 90% identical to SEQ. ID NO: 13, wherein the antibody is a glycosylated IgG antibody comprising a targeted, defined N-linked glycan profile of the Fc domain of at least one of the heavy chains with a high degree of glycan uniformity (homogeneity). The antibody can comprise a heavy chain encoded by an amino acid sequence with at least about 90% identity to SEQ ID NO: 3 and a light chain encoded by an amino acid sequence with at least about 90% identity to SEQ ID NO: 5. The glycosylation profile can be an N-linked GlcNAc2Man3GlcNAc2 (GnGn) core glycan. (Man=Mannose, GlcNAc=N-acetylglucosamine). In some aspects, the antibody does not contribute to antibody-dependent enhancement (ADE) of virus infection. In an alternative of the aspects, the antibody does not contribute to antibody-dependent enhancement (ADE) of dengue virus infection.

Another aspect of the present disclosure encompasses a monoclonal antibody having specificity to CHIKV E1 protein. The antibody comprises a heavy chain encoded by an amino acid sequence with at least about 90% identity to SEQ ID NO: 7; and a light chain encoded by an amino acid sequence with at least about 90% identity to SEQ ID NO: 9, wherein the antibody is a glycosylated IgG antibody comprising a targeted, defined N-linked glycan profile of the Fc domain of at least one of the heavy chains with a high degree of glycan uniformity (homogeneity). The antibody can comprise a heavy chain encoded by an amino acid sequence with at least about 90% identity to SEQ ID NO: 15; and a light chain comprising an amino acid sequence at least 90% identical to SEQ. ID NO: 17. The glycosylation profile can be an N-linked GlcNAc2Man3GlcNAc2 (GnGn) core glycan. (Man=Mannose, GlcNAc=N-acetylglucosamine). In some aspects, the antibody does not contribute to antibody-dependent enhancement (ADE) of virus infection. In an alternative of the aspects, the antibody does not contribute to antibody-dependent enhancement (ADE) of dengue virus infection.

An additional aspect of the present disclosure encompasses a method of producing monoclonal antibody having specificity to CHIKV E1 or E2 protein. The method comprises introducing into a plant expression system, one or more vectors for the expression of an anti-CHKV E1 or E2 antibody wherein the plant expression system is genetically engineered to produce a defined N-glycan profile. The plant expression system can be ΔXFT N. benthamiana. The glycan profile can be an N-linked GlcNAc2Man3GlcNAc2 (GnGn) core glycan. (Man-Mannose, GlcNAc=N-acetylglucosamine). The vector can be a geminiviral vector. The vector can comprise a nucleic acid sequence at least 90% identical to SEQ ID NO: 2 and a nucleic acid sequence at least 90% identical to SEQ ID NO: 4. Alternatively, the vector comprises a nucleic acid sequence at least 90% identical to SEQ ID NO: 6 and a nucleic acid sequence at least 90% identical to SEQ ID NO: 8. The vector can also comprise a nucleic acid sequence at least 90% identical to SEQ ID NO: 10 and a nucleic acid sequence at least 90% identical to SEQ ID NO: 12 or a nucleic acid sequence at least 90% identical to SEQ ID NO: 14 and a nucleic acid sequence at least 90% identical to SEQ ID NO: 16. In some aspects, the antibody does not contribute to antibody-dependent enhancement (ADE) of virus infection. In an alternative of the aspects, the antibody does not contribute to antibody-dependent enhancement (ADE) of dengue virus infection.

Yet an additional aspect of the present disclosure encompasses a method of treating and preventing infections by CHIKV in a subject in need thereof, the method comprising administering antibodies having specificity to CHIKV E1 or E2 protein to the subject. In some aspects, the antibody does not contribute to antibody-dependent enhancement (ADE) of virus infection. In an alternative of the aspects, the antibody does not contribute to antibody-dependent enhancement (ADE) of dengue virus infection.

Another additional aspect of the present disclosure encompasses a monoclonal antibody having specificity to Chikungunya virus (CHIKV) E1 or E2 protein. The antibody is a glycosylated IgG antibody comprising a targeted, defined N-linked glycan profile of the Fc domain of at least one of the heavy chains of the antibody with a high degree of glycan uniformity (homogeneity). Further, the antibody does not contribute to antibody-dependent enhancement of virus infection.

One aspect of the present disclosure encompasses a kit comprising one or more of the anti-CHICKV monoclonal antibodies of claim 1, 16, or 19, one or more composition of claim 15, one or more plant, plant fragment, or plant cell of claim 12, or combinations thereof. The antibody can comprise a heavy chain comprising an amino acid sequence at least 90% identical to SEQ. ID NO: 3; and a light chain comprising an amino acid sequence at least 90% identical to SEQ. ID NO: 5. The antibody can also comprise a heavy chain variable domain encoded by an amino acid sequence with at least about 90% identity to SEQ ID NO: 11; and a light chain variable domain encoded by an amino acid sequence with at least about 90% identity to SEQ ID NO: 13. In some aspects, a heavy chain comprising an amino acid sequence at least 90% identical to SEQ. ID NO: 7; and a light chain comprising an amino acid sequence at least 90% identical to SEQ. ID NO: 9. In other aspects, the antibody comprises a heavy chain variable domain encoded by an amino acid sequence with at least about 90% identity to SEQ ID NO: 15; and a light chain variable domain encoded by an amino acid sequence with at least about 90% identity to SEQ ID NO: 17. In some aspects, the antibody does not contribute to antibody-dependent enhancement (ADE) of dengue virus infection.

### REFERENCE TO COLOR FIGURES

The application file contains at least one figure executed in color. Copies of this patent application publication with color figures will be provided by the Office upon request and payment of the necessary fee.

### BRIEF DESCRIPTION OF THE FIGURES

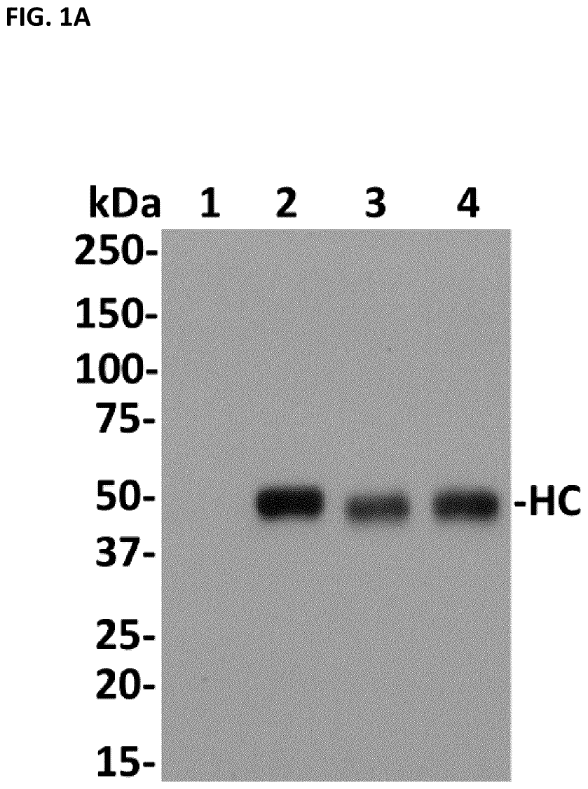

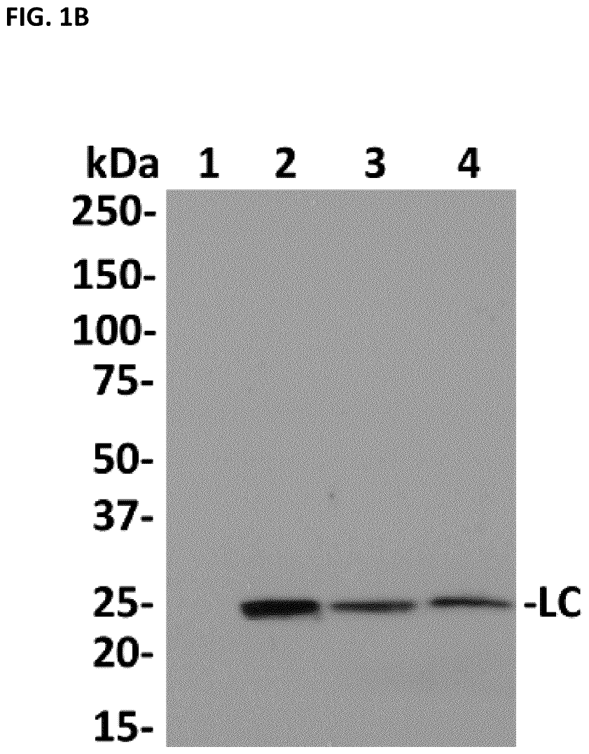

FIG. 1A. Western blot analysis of plant-produced CHKV 166. CHKV 166 was extracted from WT or ΔXFT N. benthamiana leaves, separated on SDS-PAGE gels under reducing and blotted onto PVDF membranes. A goat anti-human gamma chain antibody was incubated with the membranes to detect heavy chain or light chain. Lane 1, protein sample extracted from non-infiltrated leaves; Lane 2, mammalian cell-produced anti-WNV E16 mAb as a reference standard; Lane 3, sample from leaves of WT plants infiltrated with CHKV 166 constructs; Lane 4, sample from leaves of ΔXFT plants infiltrated with CHKV 166 constructs. HC: heavy chain, LC: light chain, (HL)₂: assembled mAb with 2 light and heavy chains.

FIG. 1B. Western blot analysis of plant-produced CHKV 166. CHKV 166 was extracted from WT or ΔXFT N. benthamiana leaves, separated on SDS-PAGE gels under reducing and blotted onto PVDF membranes. A goat anti-human kappa chain antibody was incubated with the membranes to detect heavy chain or light chain. Lane 1, protein sample extracted from non-infiltrated leaves; Lane 2, mammalian cell-produced anti-WNV E16 mAb as a reference standard; Lane 3, sample from leaves of WT plants infiltrated with CHKV 166 constructs; Lane 4, sample from leaves of ΔXFT plants infiltrated with CHKV 166 constructs. HC: heavy chain, LC: light chain, (HL)₂: assembled mAb with 2 light and heavy chains.

FIG. 1C. Western blot analysis of plant-produced CHKV 166. CHKV 166 was extracted from WT or ΔXFT N. benthamiana leaves, separated on SDS-PAGE gels under non-reducing (c) conditions and blotted onto PVDF membranes. A goat anti-human kappa chain antibody was incubated with the membranes to detect heavy chain or light chain. Lane 1, protein sample extracted from non-infiltrated leaves; Lane 2, mammalian cell-produced anti-WNV E16 mAb as a reference standard; Lane 3, sample from leaves of WT plants infiltrated with CHKV 166 constructs; Lane 4, sample from leaves of ΔXFT plants infiltrated with CHKV 166 constructs. HC: heavy chain, LC: light chain, (HL)₂: assembled mAb with 2 light and heavy chains.

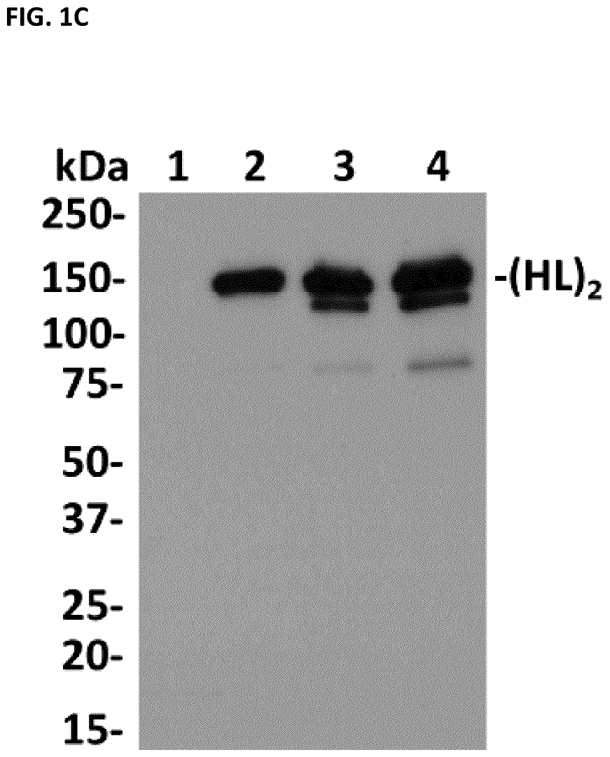

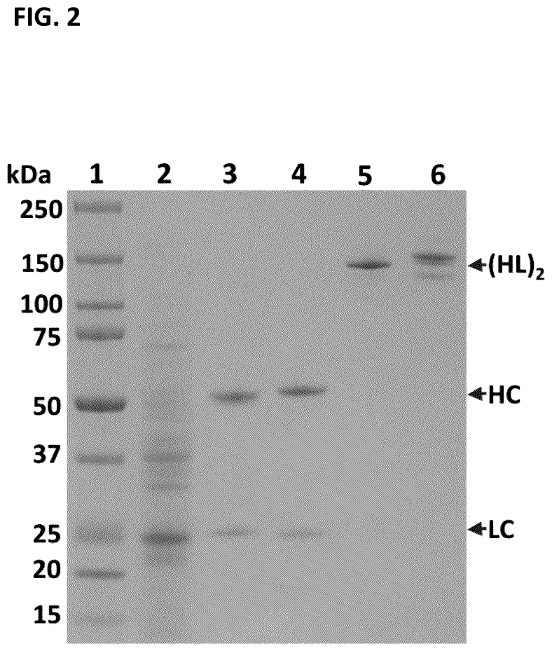

FIG. 2. SDS-PAGE gel showing purification of CHKV 166 from N. benthamiana leaves. Total soluble proteins were extracted from plants and CHKV 166 was purified and analyzed on a 4-20% gradient SDS-PAGE gel under reducing (Lanes 1-4) or non-reducing (Lanes 5 and 6) conditions and visualized with Coomassie stain. Lane 1, protein molecular weight marker; lane 2, leaf soluble proteins after low pH precipitation; lanes 3 and 5, CHKV 166 purified from leaves; lanes 4 and 6, mammalian cell-produced anti-WNV E16 mAb as a reference standard. HC: heavy chain, LC: light chain, (HL)₂: assembled mAb with 2 light and heavy chains. One representative of several independent experiments is shown.

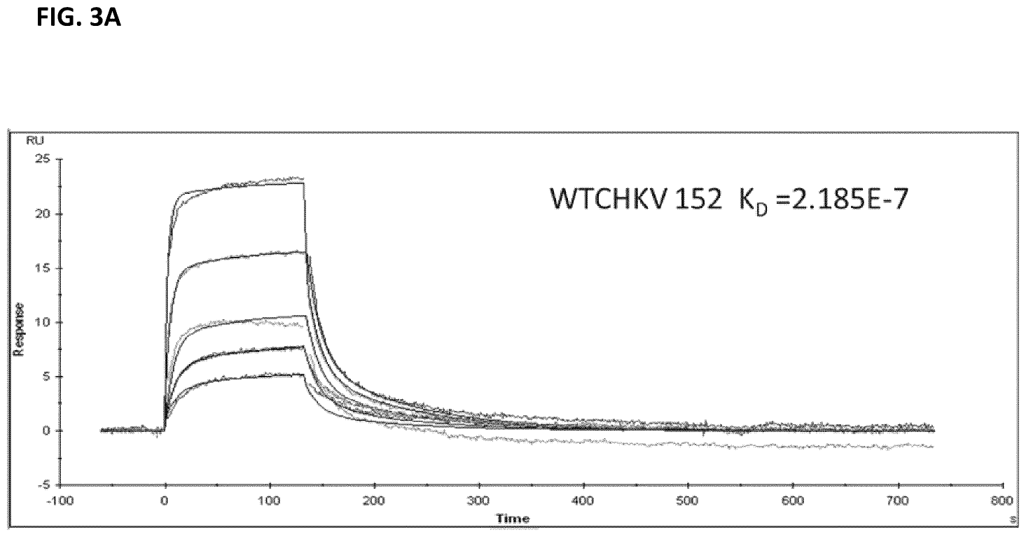

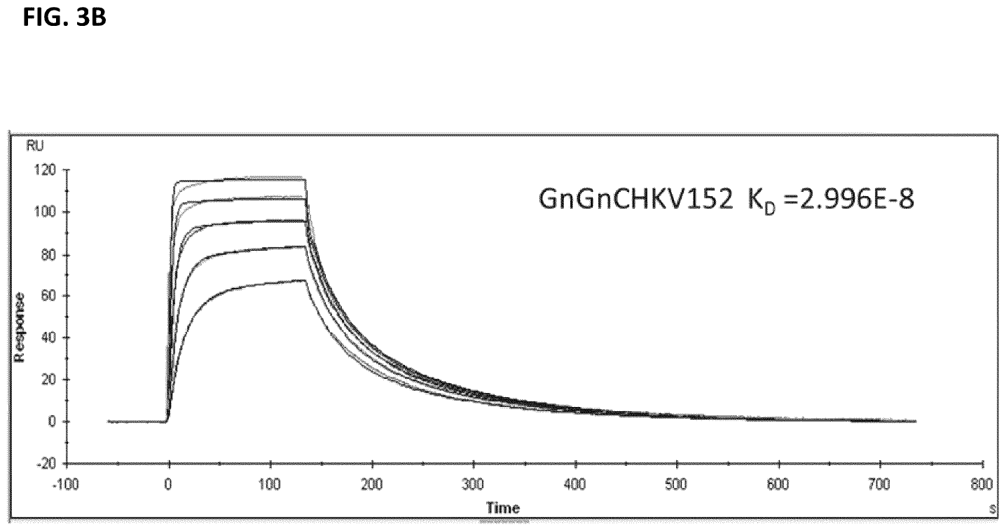

FIG. 3A. SPR analysis of binding affinity and kinetics of CHKV 152 mAb for human FcγRIIIA. Soluble human FcγRIIIA was injected over WTCHKV 152 captured to the CM-5 biosensor chip. Binding responses (colored lines) were analyzed and fitted by a two-state binding interaction model (black lines). A representative set of SPR binding curves from several independent experiments is shown.

FIG. 3B. SPR analysis of binding affinity and kinetics of CHKV 152 mAb for human FcγRIIIA. Soluble human FcγRIIIA was injected over GnGnCHKV 152 captured to the CM-5 biosensor chip. Binding responses (colored lines) were analyzed and fitted by a two-state binding interaction model (black lines). A representative set of SPR binding curves from several independent experiments is shown.

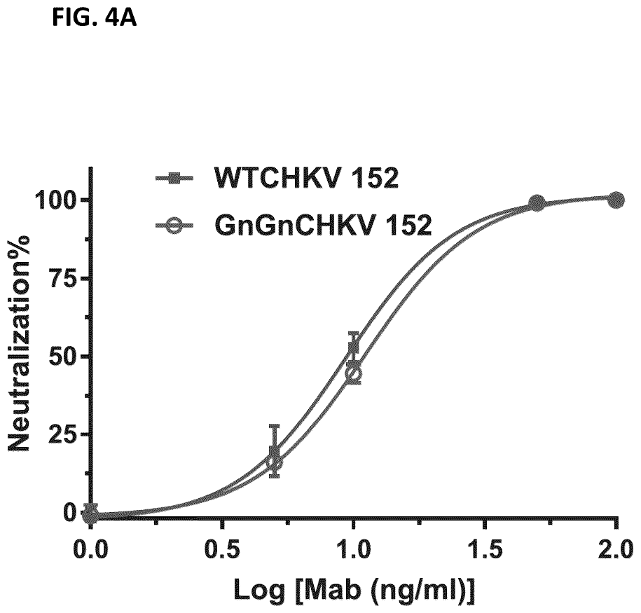

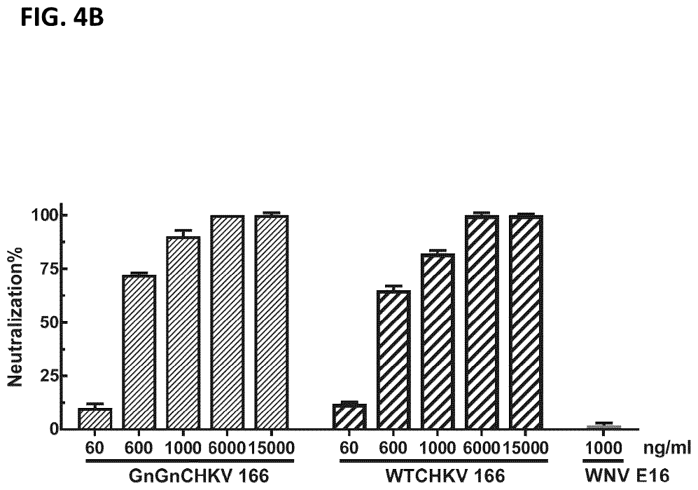

FIG. 4A. Graph of the neutralizing activity of CHKV 152 and CHKV 166. Dilutions of WTCHKV 152 and GnGnCHKV 152 were co-incubated with 100 PFU of CHIKV and Vero cells for 3 days. Plaques were counted and percent neutralization and EC₅₀ were calculated. Experiments were performed twice with technical triplicates for each sample. Bars represent the standard deviation (SD) of the mean.

FIG. 4B. Bar graph of the neutralizing activity of CHKV 152 and CHKV 166. Dilutions dilutions of WTCHKV 166, GnGnCHKV 166 or a plant-produced anti-WNV E16 mAb (WNV E16, negative control) were co-incubated with 100 PFU of CHIKV and Vero cells for 3 days. Plaques were counted and percent neutralization and EC₅₀ were calculated. Experiments were performed twice with technical triplicates for each sample. Bars represent the standard deviation (SD) of the mean.

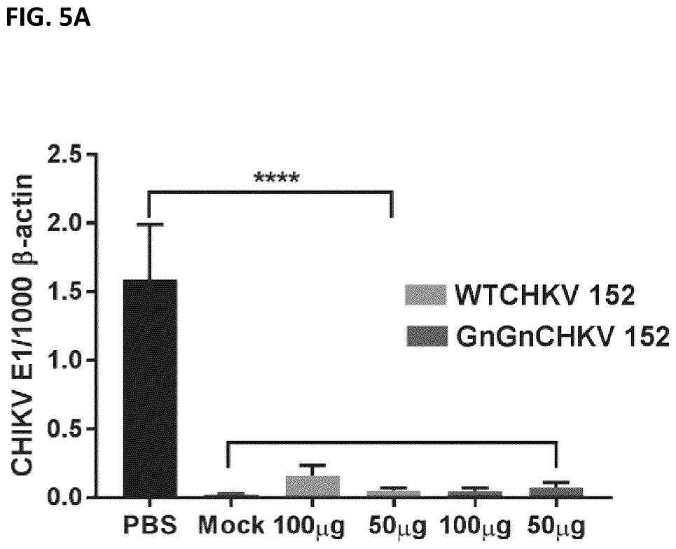

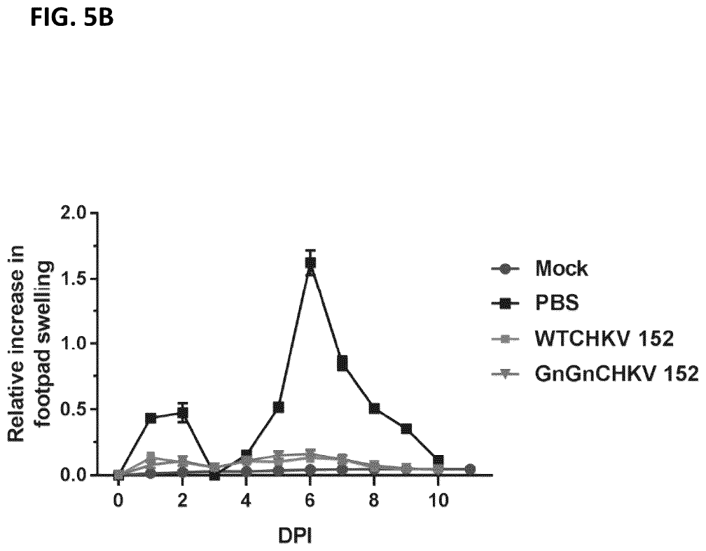

FIG. 5A. Bar graph showing prophylactic treatment with plant-produced CHKV 152 reduces CHIKV viremia in mice. Wild-type C57BL/6J mice (N=6 per group) were subcutaneously infected with 1×10⁵ PFUs of CHIKV or PBS (mock) and treated intraperitoneally with 50-100 μg of indicated CHKV 152 glycovariants or PBS (negative control) 24 hr before infection. Viral load in blood at day 2 dpi is presented as a ratio of CHIKV E1 copy number per 1000 copy of cellular β-actin.

FIG. 5B. Graph of prophylactic treatment with plant-produced CHKV 152 eliminates footpad swelling in mice. Wild-type C57BL/6J mice (N=6 per group) were subcutaneously infected with 1×10⁵ PFUs of CHIKV or PBS (mock) and treated intraperitoneally with 50 μg of indicated CHKV 152 glycovariants or PBS (negative control) 24 hr before infection. Footpad swelling was measured daily from 0 to 10 dpi and expressed as the relative increase in footpad compared to pre-infection.

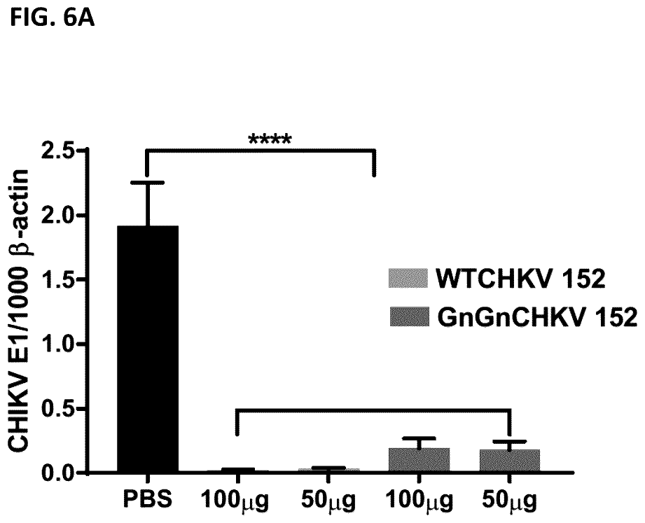

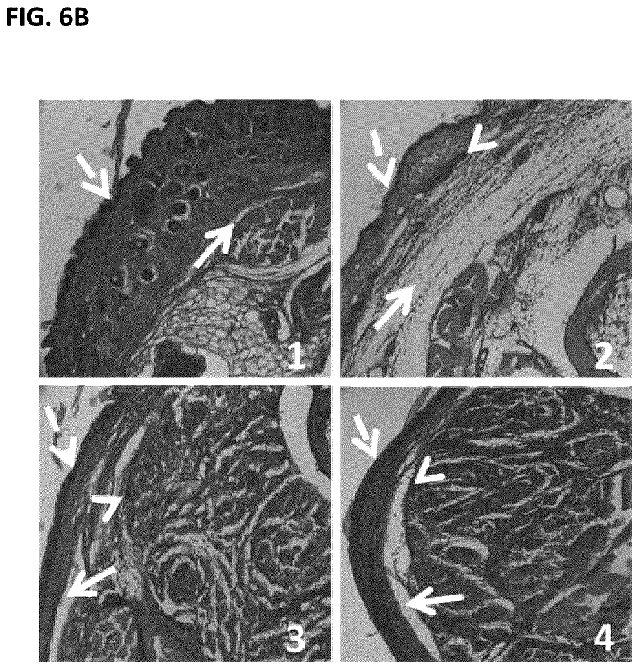

FIG. 6A. Bar graph of post-exposure treatment with plant-derived CHKV 152 reduces CHIKV viremia. Wild-type C57BL/6J mice (N=6 per group) were subcutaneously infected with 1×10⁵ PFUs of CHIKV or PBS (mock) and treated intraperitoneally with 50-100 μg of indicated CHKV 152 glycovariants or PBS (negative control) 12 hr after infection. Viral load in blood at day 2 dpi is presented as a ratio of CHIKV E1 copy number per 1000 copy of cellular β-actin.

FIG. 6B. Photomicrograph images showing that post-exposure treatment with plant-derived CHKV 152 reduces tissue damage. Wild-type C57BL/6J mice (N=6 per group) were subcutaneously infected with 1×10⁵ PFUs of CHIKV or PBS (mock) and treated intraperitoneally with 100 μg of indicated CHKV 152 glycovariants or PBS (negative control) 12 hr after infection. Pathological histology of footpad at 6 dpi. Panel 1: mock (noninfected); Panel 2: Infected, treated with saline; Panel 3: Infected, treated with WTCHKV 152; Panel 4: infected, treated with GnGnCHKV 152. Damage by CHIKV infection and the therapeutic effect of CHKV 152 on connective tissue (arrow), subcutaneous tissue (dashed arrow) are shown. Leukocyte infiltration is also shown (arrowhead).

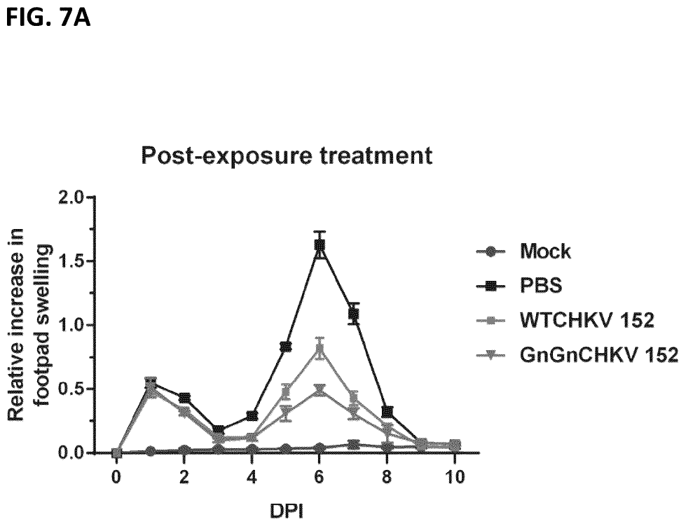

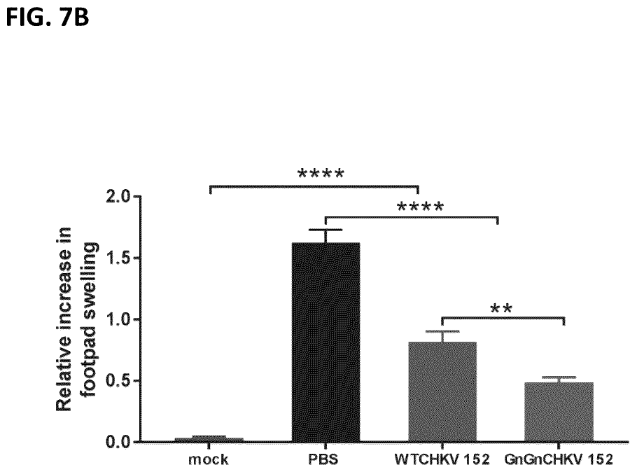

FIG. 7A. Graph showing that GnGnCHKV 152 is more potent in reducing footpad swelling in CHIKV-infected mice than WTCHKV 152. Wild-type C57BL/6J mice (N=6 per group) were subcutaneously infected with 1×10⁵ PFUs of CHIKV or PBS (mock) and treated intraperitoneally with 50 μg of indicated CHKV 152 glycovariants or PBS (negative control) 12 hr after infection. Footpad swelling was measured daily from 0 to 10 dpi and expressed as the relative increase in footpad compared to pre-infection. **** and ** indicate p values<0.0001 and p<0.0015, respectively.

FIG. 7B. Bar plot showing that GnGnCHKV 152 is more potent in reducing footpad swelling in CHIKV-infected mice than WTCHKV 152. Wild-type C57BL/6J mice (N=6 per group) were subcutaneously infected with 1×10⁵ PFUs of CHIKV or PBS (mock) and treated intraperitoneally with 50 μg of indicated CHKV 152 glycovariants or PBS (negative control) 12 hr after infection. Footpad swelling was measured daily from 0 to 10 dpi and expressed as the relative increase in footpad compared to pre-infection. ** ** and ** indicate p values<0.0001 and p<0.0015, respectively.

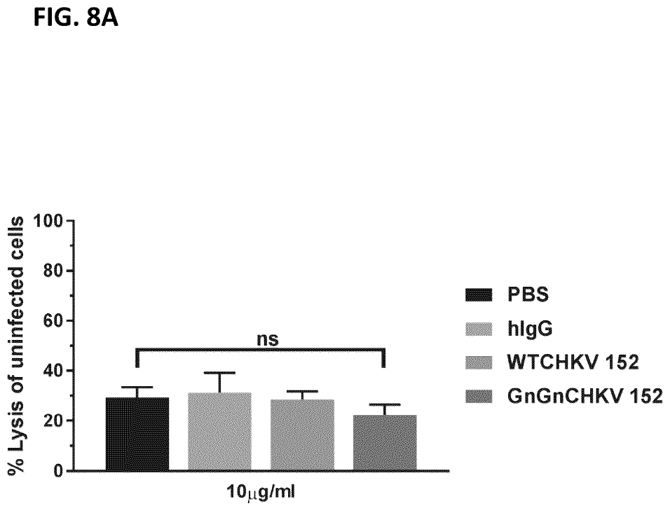

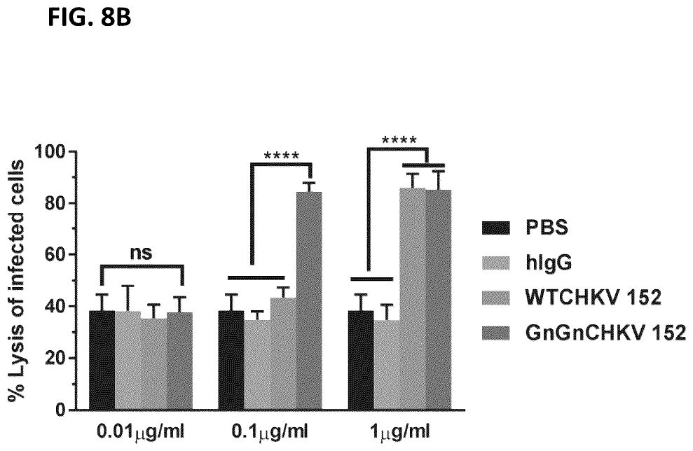

FIG. 8A. Bar plot showing that GnGnCHKV 152 has more potent natural killer cell-mediated ADCC activity against CHIKV infected cells than WTCHKV 152. Uninfected Vero cells were loaded with Calcein AM and then incubated with the indicated concentrations of CHKV 152 glycovariants or an isotype control IgG (hIgG, negative control). NK cells were then added to Vero cells at an E/T ratio of 1.25:1. Vero cell lysis by NK cells is expressed by the % of non-spontaneous VERO cell lysis in the presence of NK cells/maximal lysis by detergent treatment.

FIG. 8B. Bar plot showing that GnGnCHKV 152 has more potent natural killer cell-mediated ADCC activity against CHIKV infected cells than WTCHKV 152. CHIKV-infected Vero cells were loaded with Calcein AM and then incubated with the indicated concentrations of CHKV 152 glycovariants or an isotype control IgG (hIgG, negative control). NK cells were then added to Vero cells at an E/T ratio of 1.25:1. Vero cell lysis by NK cells is expressed by the % of non-spontaneous VERO cell lysis in the presence of NK cells/maximal lysis by detergent treatment. **** indicates p value<0.0001.

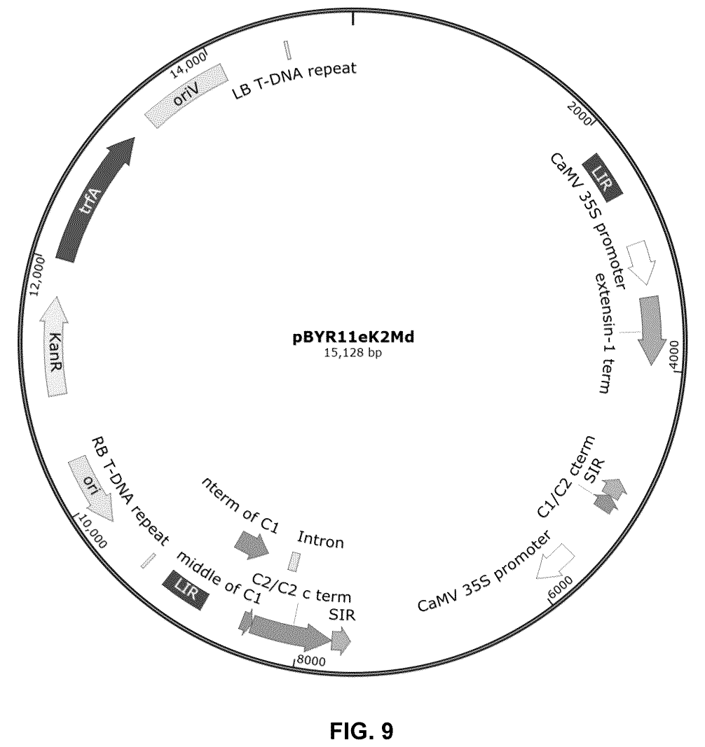

FIG. 9. Map of gemini virus-based vector for expressing mAbs in plants. LIR: long intergenic region of BeYDV genome; SIR: short intergenic region of BeYDV genome; C1/C2: BeYDV ORFs C1 and C2, encoding replication initiation protein (Rep) and RepA; LB and RB, the left and right borders of the T-DNA.

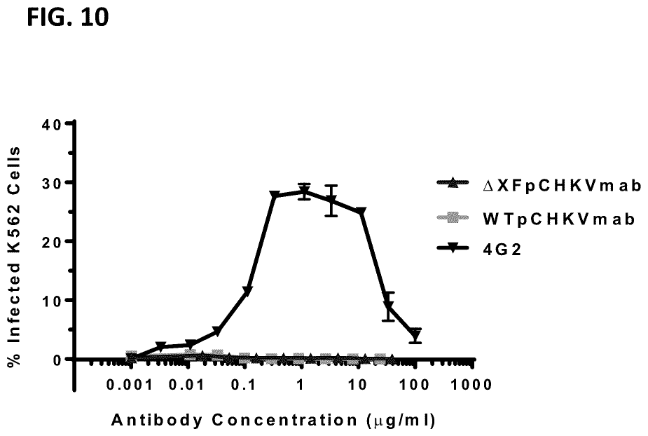

FIG. 10. Graph addressing antibody-dependent enhancement of CHKVmabs for DENV infection. Serial dilutions of CHKVmabs or 4G2 (positive control mAb) were mixed with DENV-2 and then incubated with FcγRIIa+K562 cells. Cells were fixed, permeabilized and stained with an anti-DENV E antibody and analyzed by flow cytometry.

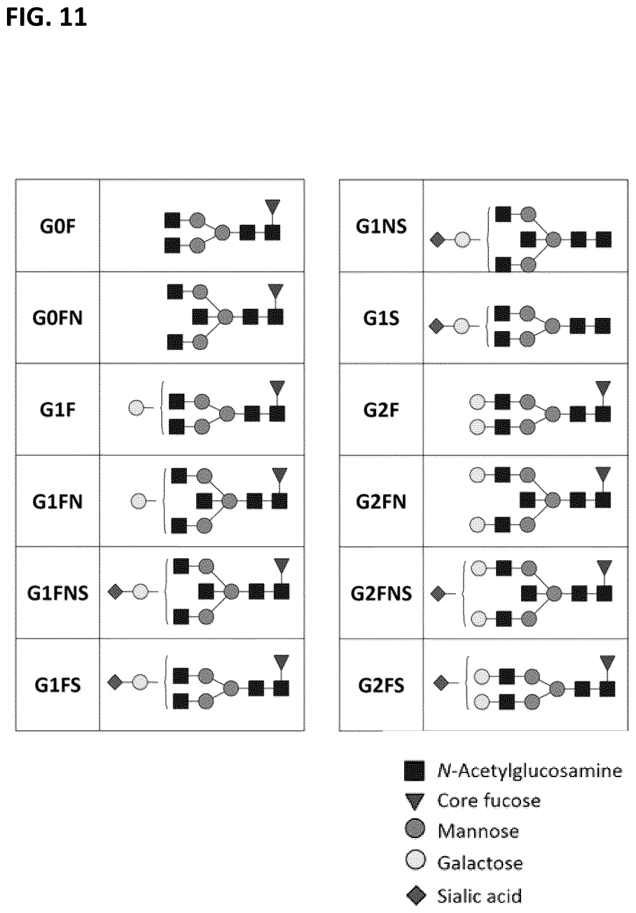

FIG. 11 depicts prominent glycan structures attached to the conserved N-glycosylation site on each of the two IgG heavy chains.

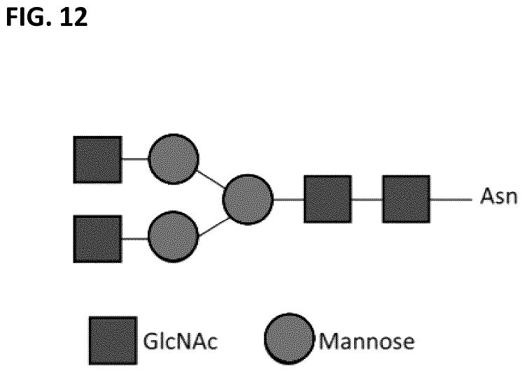

FIG. 12 depicts the N-linked GlcNAc2Man3GlcNAc2 (GnGn; G0) glycosylation profile of the antibody. (GlcNAc=N-acetylglucosamine)

### DETAILED DESCRIPTION

The present disclosure encompasses glycosylated antibodies comprising targeted, defined, and uniform glycosylation profiles, and methods of producing the antibodies. The methods were used to produce anti-CHIKV glycosylated antibodies having enhanced effector functions. Importantly, the antibodies exhibit improved effector functions, all while retaining the binding activity and affinity of the antibody to a specific target.

### I. Composition

One aspect of the present disclosure encompasses a monoclonal antibody. In an aspect, antibodies useful herein include those antibodies which have been isolated, characterized, purified, are functional and have been recovered (obtained) for use in a functional therapeutic composition which is administered to a living patient.

The term “antibody” refers to an immunoglobulin molecule capable of specific binding to a target, such as a carbohydrate, polynucleotide, lipid, or polypeptide through at least one antigen recognition site. As used herein, an antibody encompasses not only intact polyclonal or monoclonal antibodies, but also fragments thereof (such as Fab, Fab′, F(ab′)2. Fv), Fc, single chain (ScFv), mutants thereof, fusion proteins comprising an antibody portion, humanized antibodies, chimeric antibodies, diabodies, linear antibodies, single chain antibodies, multispecific antibodies (e.g., bispecific antibodies) and any other modified configuration of the immunoglobulin molecule that comprises an antigen recognition site of the required specificity.

Depending on the antibody amino acid sequence of the constant domain of its heavy chains, immunoglobulins can be assigned to different classes. In mammals, heavy-chains are classified as alpha, delta, epsilon, gamma, or mu, and define the antibody's isotype as IgA, IgD, IgE, IgG, and IgM, respectively. Several of these isotypes may be further divided into subclasses (isotypes), e.g., IgG1, IgG2, IgG3, IgG4, IgA1 and IgA2. Light chains are classified as kappa and lambda.

Antibodies of the instant disclosure can be of any class, such as IgG, IgA, or IgM (or sub-class thereof), or the antibody need not be of any particular class. Further, intact monoclonal antibodies and full-length monoclonal antibodies are contemplated. As long as the protein retains the ability specifically to bind its intended target, it is included within the term “antibody.” In some aspects, the antibody is an IgG antibody.

The antibodies of the disclosure can be monoclonal antibodies. “Monoclonal antibody” (mAb) refers to an antibody that is derived from a single copy or clone. A monoclonal antibody is not limited to antibodies produced through hybridoma technology. Monoclonal antibodies may be produced using e.g., hybridoma techniques well known in the art, as well as recombinant technologies, phage display technologies, synthetic technologies, plant expression systems, or combinations of such technologies and other technologies readily known in the art.

Antibodies useful herein include those that are isolated, characterized, purified, functional, and have been recovered (obtained) from a process for their preparation and thus available for use herein in a useful form in a therapeutically and medicinally sufficient amount. In some aspects, the antibodies are generated in plants as described in Section II(a) below.

Antibodies with appropriate specificity can be generated by standard techniques of immunization of mammals, forming hybridomas from the antibody-producing cells of said mammals or otherwise immortalizing them, and culturing the hybridomas or immortalized cells to assess them for the appropriate specificity. Antibodies may be generated by immunizing a human, rabbit, rat or mouse, for example, with a peptide representing an epitope encompassing a viral particle, a viral protein, or a fragment thereof.

The antibodies of the present disclosure may also be chimeric antibodies. These chimeric antibodies can involve the merging of a portion of a monoclonal antibody with antibody-producing DNA in living cells to produce a monoclonal antibody that has material from more than one species of animal. This procedure is well known in the art and any known method to produce chimeric antibodies is suitable for purposes of the present disclosure.

As used herein “humanized antibody” includes an antibody that is composed partially or fully of amino acid sequences derived from a human antibody germline by altering the sequence of an antibody having non-human complementarity determining regions (“CDR”). The simplest such alteration may consist simply of substituting the constant region of a human antibody for the murine constant region, thus resulting in a human/murine chimera which may have sufficiently low immunogenicity to be acceptable for pharmaceutical use. However, the variable region of the antibody and even the CDR can be humanized by techniques that are well known in the art. The framework regions of the variable regions can be substituted by the corresponding human framework regions leaving the non-human CDR substantially intact, or even replacing the CDR with sequences derived from a human genome. CDRs may also be randomly mutated such that binding activity and affinity is maintained or enhanced in the context of fully human germline framework regions or framework regions that are substantially human. Substantially human frameworks can have at least 90%, 95%, or 99% sequence identity with a known human framework sequence. Fully useful human antibodies can be produced in genetically modified mice whose immune systems have been altered to correspond to human immune systems. As mentioned above, it is sufficient for use in the methods of this discovery, to employ an immunologically specific fragment of the antibody, including fragments representing single chain forms.

The antibody polypeptides of the present disclosure can be in isolated form. As used herein, a polypeptide is said to be isolated when physical, mechanical or chemical methods are employed to remove the protein from cellular constituents that are normally associated with the protein. A skilled artisan can readily employ standard purification methods to obtain an isolated protein.

The antibodies of the instant disclosure are specific to CHICKV virions, CHIKV polypeptides, or fragments thereof. Chikungunya virus (CHIKV) is a positive-sense RNA virus belonging to the alphavirus family, which can be transmitted to humans by mosquitos. CHIKV is an enveloped virus enclosing a single-stranded positive sense RNA genome of an approximate 11.8 kbp. The two open reading frames (ORFs) code for 4 non-structural proteins (NS-1, 2, 3, 4) and 3 structural proteins-nucleocapsid (C) and envelope protein1 and 2 (E1 and E2). The C protein forms the central capsid core which encapsulates viral RNA. The nucleocapsid, in turn, is surrounded by the viral envelope studded with E1 and E2. The E1-E2 heterodimers are arranged into 80 trimeric spikes with a single CHIKV virion containing 240 copies of the envelope proteins on its surface. During a CHIKV infection, E2 functions in attachment and triggers receptor-mediated endocytosis. The low pH within the endosome leads to dissociation of the E1-E2 heterodimer, exposing the fusion loop of E1 and leading to a type II membrane fusion.

The antibodies can be specific to a CHICKV protein, a CHIKV virion, or fragments a CHICKV protein or CHIKV virion. In some aspects, an antibody has specificity to a nonstructural protein such as NS-1, NS2, NS3, and NS4. In other aspects, the antibody has specificity to a structural protein such as E1, E2 and C.

One aspect of the present disclosure encompasses antibodies that specifically bind the E1 protein of CHIKV. In one aspect, such an antibody is a monoclonal IgG antibody comprising a heavy chain amino acid sequence with at least about 60% identity to SEQ ID NO: 3. In some aspects, the heavy chain amino acid sequence comprises at least about 60, 65, 70, 75, 80, 85, 90, or 95% identical to SEQ. ID NO: 3. In some aspects, the heavy chain amino acid sequence comprises at least about 95, 96, 97, 98, or 99% sequence homology with SEQ ID NO: 3. In some aspects, the heavy chain amino acid sequence is encoded by SEQ ID NO: 3. In one aspect, the antibody is encoded by a heavy chain amino acid sequence that comprises at least about 60% identity to SEQ ID NO: 3. In one aspect, the light chain of the antibody is encoded by an amino acid sequence with at least about 60% identity to SEQ ID NO: 5. In some aspects, the light chain amino acid sequence comprises at least about 60, 65, 70, 75, 80, 85, 90, or 95% identical to SEQ. ID NO: 5. In some aspects, the light chain amino acid sequence comprises at least about 95, 96, 97, 98, or 99% sequence homology with SEQ ID NO:5. In some aspects, the light chain amino acid sequence is encoded by the amino acid sequence of SEQ ID NO:5. In one aspect, the antibody is encoded by a light chain amino acid sequence that comprises at least about 60% identity to SEQ ID NO: 5.

One aspect of the present disclosure encompasses antibodies that bind the E1 protein of CHIKV. In one aspect, such an antibody is a monoclonal IgG antibody comprising a heavy chain variable domain encoded by an amino acid sequence with at least about 60% identity to SEQ ID NO: 11. In some aspects, the heavy chain variable domain amino acid sequence comprises at least about 60, 65, 70, 75, 80, 85, 90, or 95% identical to SEQ. ID NO: 11. In some aspects, the heavy chain variable domain amino acid sequence comprises at least about 95, 96, 97, 98, or 99% sequence homology with SEQ ID NO: 11. In some aspects, the heavy chain variable domain amino acid sequence is encoded by SEQ ID NO: 11. In one aspect, the variable region of the heavy chain amino acid sequence comprises at least about 60% identity to SEQ ID NO: 11. In one aspect, the light chain variable region is encoded by an amino acid sequence that comprises at least about 60% identity to SEQ ID NO: 13. In some aspects, the light chain variable region amino acid sequence comprises at least about 60, 65, 70, 75, 80, 85, 90, or 95% identical to SEQ. ID NO: 13. In some aspects, the light chain variable region amino acid sequence comprises at least about 95, 96, 97, 98, or 99% sequence homology with SEQ ID NO: 13. In some aspects, the light chain variable region amino acid sequence is encoded by the amino acid sequence of SEQ ID NO: 13. In one aspect, the variable region of the light chain amino acid sequence comprises at least about 60% identity to SEQ ID NO: 13.

One aspect of the present disclosure encompasses antibodies that bind the E2 protein of CHIKV. In one aspect, such an antibody is a monoclonal IgG antibody comprising a heavy chain amino acid sequence with at least about 60% identity to SEQ ID NO: 7. In some aspects, the heavy chain amino acid sequence comprises at least about 60, 65, 70, 75, 80, 85, 90, or 95% identical to SEQ. ID NO: 7. In some aspects, the heavy chain amino acid sequence comprises at least about 95, 96, 97, 98, or 99% sequence homology with SEQ ID NO: 7. In some aspects, the heavy chain amino acid sequence is encoded by SEQ ID NO: 7. In one aspect, the heavy chain amino acid sequence comprises at least about 60% identity to SEQ ID NO: 7. In one aspect, the antibody is encoded by a light chain amino acid sequence with at least about 60% identity to ID NO: 9. In one aspect, the light chain is encoded by an amino acid sequence that comprises at least about 60% identity to SEQ ID NO: 9. In some aspects, the light chain amino acid sequence comprises at least about 60, 65, 70, 75, 80, 85, 90, or 95% identical to SEQ. ID NO: 9. In some aspects, the light chain amino acid sequence comprises at least about 95, 96, 97, 98, or 99% sequence homology with SEQ ID NO: 9. In some aspects, the light chain amino acid sequence is encoded by the amino acid sequence of SEQ ID NO: 9. In one aspect, the light chain amino acid sequence comprises at least about 60% identity to SEQ ID NO: 9.

One aspect of the present disclosure encompasses antibodies that bind the E2 protein of CHIKV. In one aspect, such an antibody is a monoclonal IgG antibody comprising a heavy chain variable domain encoded by an amino acid sequence with at least about 60% identity to SEQ ID NO: 15. In some aspects, the heavy chain variable domain amino acid sequence comprises at least about 60, 65, 70, 75, 80, 85, 90, or 95% identical to SEQ. ID NO: 15. In some aspects, the heavy chain variable domain amino acid sequence comprises at least about 95, 96, 97, 98, or 99% sequence homology with SEQ ID NO: 15. In some aspects, the heavy chain variable domain amino acid sequence is encoded by SEQ ID NO: 15. In one aspect, the variable region of the heavy chain amino acid sequence comprises at least about 60% identity to SEQ ID NO: 15. In one aspect, the light chain variable region is encoded by an amino acid sequence with at least about 60% identity to SEQ ID NO: 17. In some aspects, the light chain variable region amino acid sequence comprises at least about 60, 65, 70, 75, 80, 85, 90, or 95% identical to SEQ. ID NO: 17. In some aspects, the light chain variable region amino acid sequence comprises at least about 95, 96, 97, 98, or 99% sequence homology with SEQ ID NO: 17. In some aspects, the light chain variable region amino acid sequence is encoded by the amino acid sequence of SEQ ID NO: 17. In one aspect, the variable region of the light chain amino acid sequence comprises at least about 60% identity to SEQ ID NO: 17.

In mammalian cells hundreds of N-linked glycoforms can be detected by mass spectrometric analyses on total protein extractions. Such heterogeneity makes a controlled production of antibodies with uniform and targeted glycosylation profiles difficult and hinders batch to batch consistency, an important requirement of the regulatory authorities. Moreover, glycosylation heterogeneity impedes studies to determine the impact of individual glycoforms to a protein's function.

The glycans attached to glycoproteins contain a variety of sugar residues linked in linear or branched structures that can assume many different conformations. These glycans can play a fundamental role in promoting correct protein folding and assembly and, as a consequence, enhance protein stability. They may also contain targeting information, or may be directly involved in protein recognition. The N-linked glycosylation mechanisms in mammalian and plant systems have been conserved during evolution. However, differences are observed in the final steps of oligosaccharide trimming and glycan modification in the Golgi apparatus. In contrast to bacteria, having no N-linked glycans, and yeast, having polymannose glycans, plants produce glycoprotein multimers with complex N-linked glycans having a core substituted by two N-acetylglucosamine (GlcNAc) residues. These glycoprotein multimers are also observed in mammals. Plant and animal glycopolypeptide multimers contain different terminal carbohydrates that are directly linked to the outer branches of the oligosaccharides present. Animal glycopolypeptide multimers, including mammalian glycopolypeptide multimers, have sialic acid present as a terminal carbohydrate residue, while plant glycopolypeptide multimers do not. The terminal core is substituted by 1,2-linked xylose (Xyl) and α1,3-linked core fucose (Fuc) instead of α1,6-linked core fucose as occur in mammals. Furthermore, plant glycoprotein multimers lack the characteristic galactose (Gal)- and sialic acid-containing complex N-glycans (N-acetylneuraminic-α2-6/3Galβ1-4) found in mammals.

IgG antibodies comprise a single glycosylation site for N-linked glycosylation. As such, antibodies of the instant disclosure comprise a single glycan molecule attached to an Fc region on the heavy chain of the antibody. Each intact antibody comprises two heavy chains, and therefore two glycosylation sites. In one aspect, one glycosylation site is glycosylated. In another aspect, two glycosylation sites are glycosylated. In one alternative of the aspect, both glycosylation sites are glycosylated with the same glycan structure. In other aspects, each glycosylation site is glycosylated with a different glycan molecule. Prominent glycan structures attached to the conserved N-glycosylation site on each of the two IgG heavy chains are shown in FIG. 11.

In the instant disclosure, the antibody comprises a uniform and targeted N-linked glycosylation profile of the Fc domain of at least one of the heavy chains with a high degree of glycan uniformity (homogeneity). An antibody having a targeted glycosylation profile comprises a pre-selected glycosylation profile of the antibody. As used interchangeably herein, “uniformity” and “homogeneity” refer to the degree of sameness of the glycans in a glycoform of an antibody, expressed as a percentage. An N-glycan profile analysis is performed by determining the N-glycosylation of an antibody produced as described herein, which may be determined for example by mass spectrometry methods, such as liquid chromatography-electrospray ionization-mass spectrometry (LC-ESI-MS). Glycan uniformity is determined as a percentage for each different glycan present in the total, wherein for example an antibody having a mixture of N-glycans as shown in Table 1 below (see Example 4), will exhibit a relatively low percentage of each of several different glycans. In contrast, an antibody with a high degree of glycan uniformity exhibits a relatively high (at least about 80%) percentage of one glycan.

In some aspects, the glycosylation profile of the antibody is an N-linked GlcNAc2Man3GlcNAc2 (GnGn; G0) core glycan. (GlcNAc=N-acetylglucosamine) depicted in FIG. 12.

In contrast to glycosylation profiles of antibodies of the instant disclosure produced in mammalian cells, the targeted glycosylation profile of the antibodies are uniform targeted glycosylation profile is uniform. For instance, the glycosylation profile of the CHKV 152 mAb produced in mammalian cells comprises 40% GOF, 46% G1F, 11% G2F, and 3% G2FS.

For the antibodies described herein, the degree of glycan uniformity determined from the N-linked glycosylation profile determined as described above, is at least 80%, at least 85%, at least 90%, at least 95%, at least 96%, at least 97%, at least 98%, at least 99% or is 100%. Glycan Uniformity may range from about 80% to about 100%, from about 85% to about 100%, from about 90% to about 100%, from about 95% to about 100%, from about 85% to about 90%, from about 85% to about 95%, or from about 85% to any of about 96%, 97%, 98%, or 99%. In some aspects, the antibody Fc domain comprises a targeted N-linked glycosylation profile on one of the heavy chains with a high degree of glycan uniformity (homogeneity). In other aspects, the antibody Fc domain comprises a targeted N-linked glycosylation profile the two heavy chains with a high degree of glycan uniformity (homogeneity). It should therefore be understood that, as used herein the term “a high degree of glycan uniformity” refers to a glycan uniformity of at least about 80% but and also to any of the uniformity percentages and percentage ranges listed in the foregoing.

One of the challenges for mAb therapeutics against viral infections is the increased risk of infection by other viruses in treated patients via the mechanism of antibody-dependent enhancement of infection (ADE). For example, cross-reactive but sub-neutralizing antibodies against one serotype of DENV from a previous infection can form complexes with another serotype of DENV during a secondary infection to promote viral infection of FccR-bearing myeloid cells, predisposing patients to develop the more severe dengue haemorrhagic fever/dengue shock syndrome through ADE. As shown in FIG. 10, antibodies of the present disclosure do not contribute to antibody-dependent enhancement (ADE) of virus infection. In an alternative of the aspects, the antibodies do not contribute to antibody-dependent enhancement (ADE) of dengue virus infection.

### II. Methods

### (a) Method of Producing

The monoclonal antibodies comprise a targeted N-glycosylation profile. A protein comprising a targeted glycosylation profile is a protein glycosylated with one or more glycans having a predefined structure. The targeted glycosylation profile can be generated using a protein expression system comprising a glycosylation machinery controlled to synthesize proteins glycosylated with one or more defined glycosylation profile.

In some aspects, the monoclonal antibodies are produced in plants. Plants have been demonstrated to be an attractive alternative to mammalian cell cultures as a system for mAb development and production. The benefits of using plants for antibody production include large scale production, reduced costs for production, maintenance and delivery, as well as eliminating the risk of the resultant product containing possibly harmful contaminants such as viruses or prions that are pathogenic to humans and other mammals. Plants, like other heterologous expression systems including mammalian cells, bacteria, yeast, and insects, exhibit differences in glycosylation.

As explained above in Section I(a), hundreds of N-linked glycoforms can be detected in mammalian cells making controlled production of antibodies with homogenous glycosylation profiles difficult, hindering the consistency required of the regulatory authorities, and impeding studies on the impact of individual glycoforms to a protein's function. Another advantage of using plants for the production of glycoproteins is that they are highly amenable to glycoengineering, which allows the generation of proteins with targeted tailor-made N-glycans. This is particularly relevant to mAbs since it is well known that the glycosylation profile on the single N-glycosylation site in the Fc domain of IgG antibodies significantly impacts mAb activities. For example, the targeted removal of core fucose residues from complex N-glycans in mammalian IgGs significantly increases Fc gamma receptor (FcγR) III binding and subsequent effector functions.

Any plant capable of producing a targeted glycosylation profile of the antibodies of the instant disclosure can be used in a method of the disclosure. The plant can be naturally capable of producing the desired glycosylation profile. Alternatively, the plant is genetically engineered to introduce or remove glycosylation activities to tailor make glycosylation. Solanaceous species within the genus Nicotiana, specifically Nicotiana tabacum (tobacco) and Nicotiana benthamiana (Australian variety), are recognized as effective hosts in Molecular Farming. Advantageous features include (i) ease of cultivation and high biomass; (ii) availability of genetic tools for trait manipulation; (iii) amenability to plant breeding techniques and molecular manipulation; and (iv) non-food status, which minimizes the possibility of contamination of the food supply with industrial designated products.

In some aspects, the antibodies of the instant disclosure are produced in N. benthamiana. N. benthamiana is particularly well suited for producing recombinant proteins. A significant aspect in N. benthamiana is that it contains a natural insertion in the RNA-dependent RNA polymerase 1 gene, which leads to a reduced level of gene silencing. This feature allows the efficient utilization of transient expression vectors, enabling the rapid production (4-10 days) of high value proteins (hormones, enzymes, antibodies) by transient transfection, e.g., using agro-infiltration. Furthermore, N. benthamiana lines have been established which permit the production of proteins, including mAbs, with customized N-glycan structures and thus superior biological activities. See, for example, Niemer et al., Nicotiana benthamiana cathepsin B displays distinct enzymatic features which differ from its human relative and aleurain-like protease, Biochimie, Volume 122, March 2016, Pages 119-125; and Strasser et al., Controlled glycosylation of plant-produced recombinant proteins, Curr. Opin. Biotechnol., 30 (2014), pp. 95-100. In some aspects, the plant used to produce the instant antibodies is the N. benthamiana ΔXFT line.

Methods of transforming plants and introducing nucleic acid sequences encoding the antibodies are well known in the art. In some aspects, antibody polypeptides are produced in a plant using an agrobacterial transformation vector that launches a viral vector encoding the polypeptide of interest. In some aspects, the viral vector is a geminiviral vector.

In some aspects, the vector comprises a nucleic acid sequence at least 90% identical to SEQ ID NO: 2 and a nucleic acid sequence at least 90% identical to SEQ ID NO: 4. In other aspects, the vector comprises a nucleic acid sequence at least 90% identical to SEQ ID NO: 6 and a nucleic acid sequence at least 90% identical to SEQ ID NO: 8. In yet other aspects, the vector comprises a nucleic acid sequence at least 90% identical to SEQ ID NO: 10 and a nucleic acid sequence at least 90% identical to SEQ ID NO: 12. In additional aspects, the vector comprises a nucleic acid sequence at least 90% identical to SEQ ID NO: 14 and a nucleic acid sequence at least 90% identical to SEQ ID NO: 16.

Once produced in a plant, any suitable method can be used to partially or completely isolate an expressed antibody from plant material. As discussed above, a wide range of fractionation and separation procedures are known for purifying substances from plant tissues or fluids.

### (b) Method of Treating

One aspect of the present disclosure encompasses a method of treating and preventing infections by CHIKV in a subject in need thereof. The method comprises administering antibodies having specificity to CHIKV E1 or E2 protein to the subject. In some aspects, the antibody does not contribute to antibody-dependent enhancement of virus infection. The antibodies can be as described above in Section I. The antibodies of the present disclosure may be part of a combination therapy. The disclosure also encompasses use of any of the antibodies disclosed herein for treating and preventing infections by CHIKV in a subject.

In some aspects, antibodies of the present disclosure do not contribute to antibody-dependent enhancement (ADE) of virus infection. In an alternative of the aspects, the antibodies do not contribute to antibody-dependent enhancement (ADE) of dengue virus infection.

The subject can be a human, a livestock animal, a companion animal, a lab animal, or a zoological animal. In one aspect, the subject may be a rodent, e.g. a mouse, a rat, a guinea pig, etc. Non-limiting examples of suitable livestock animals may include pigs, cows, horses, goats, sheep, llamas and alpacas. Non-limiting examples of companion animals may include pets such as dogs, cats, rabbits, and birds. As used herein, a “zoological animal” refers to an animal that may be found in a zoo. Such animals may include non-human primates, large cats, wolves, and bears. Non-limiting examples of a laboratory animal may include rodents, canines, felines, and non-human primates. Non-limiting examples of rodents may include mice, rats, guinea pigs, etc. In some aspects the subject is a human subject.

In certain aspects, a pharmacologically effective amount of an antibody of the disclosure, including immunologically reactive fragments, may be administered to a subject. Administration is performed using standard effective techniques, include peripherally (i.e. not by administration into the central nervous system) or locally to the central nervous system. Peripheral administration includes but is not limited to intravenous, intraperitoneal, subcutaneous, pulmonary, transdermal, intramuscular, intranasal, buccal, sublingual, or suppository administration. Local administration, including directly into the central nervous system (CNS) includes but is not limited to via a lumbar, intraventricular or intraparenchymal catheter or using a surgically implanted controlled release formulation.

Pharmaceutical compositions for effective administration are deliberately designed to be appropriate for the selected mode of administration, and pharmaceutically acceptable excipients such as compatible dispersing agents, buffers, surfactants, preservatives, solubilizing agents, isotonicity agents, stabilizing agents and the like are used as appropriate. Remington's Pharmaceutical Sciences, Mack Publishing Co., Easton Pa., 16Ed ISBN: 0-912734-04-3, latest edition, incorporated herein by reference in its entirety, provides a compendium of formulation techniques as are generally known to practitioners. It may be particularly useful to alter the solubility characteristics of the antibodies useful in this discovery, making them more lipophilic, for example, by encapsulating them in liposomes or by blocking polar groups.

Effective peripheral systemic delivery by intravenous or intraperitoneal or subcutaneous injection can be a method of administration to a living patient. Suitable vehicles for such injections are straightforward. In addition, however, administration may also be effected through the mucosal membranes by means of nasal aerosols or suppositories. Suitable formulations for such modes of administration are well known and typically include surfactants that facilitate cross-membrane transfer. Such surfactants are often derived from steroids or are cationic lipids, such as N-[1-(2,3-dioleoyl) propyl]-N, N, N-trimethyl ammonium chloride (DOTMA) or various compounds such as cholesterol hemisuccinate, phosphatidyl glycerols and the like.

The concentration of antibody in formulations to be administered is an effective amount and ranges from as low as about 0.1% by weight to as much as about 15 or about 20% by weight and will be selected primarily based on fluid volumes, viscosities, and so forth, in accordance with the particular mode of administration selected if desired. A typical composition for injection to a living patient could be made up to contain 1 mL sterile buffered water of phosphate buffered saline and about 1-1000 mg of any one of or a combination of the humanized antibody of the present discovery. The formulation could be sterile filtered after making the formulation, or otherwise made microbiologically acceptable. A typical composition for intravenous infusion could have volumes between 1-250 mL of fluid, such as sterile Ringer's solution, and 1-100 mg per ml, or more in anti-tau antibody concentration. Therapeutic agents of the discovery can be frozen or lyophilized for storage and reconstituted in a suitable sterile carrier prior to use. Lyophilization and reconstitution may lead to varying degrees of antibody activity loss (e.g. with conventional immune globulins, IgM antibodies tend to have greater activity loss than IgG antibodies). Dosages administered are effective dosages and may have to be adjusted to compensate. The pH of the formulations generally pharmaceutical grade quality, will be selected to balance antibody stability (chemical and physical) and comfort to the patient when administered. Generally, a pH between 4 and 8 is tolerated. Doses will vary from individual to individual based on size, weight, and other physiobiological characteristics of the individual receiving the successful administration.

As used herein, the term “effective amount” means an amount of a substance such as a compound that leads to measurable and beneficial effects for the subject administered the substance, i.e., significant efficacy. The effective amount or dose of compound administered according to this discovery will be determined by the circumstances surrounding the case, including the compound administered, the route of administration, the status of the symptoms being treated and similar patient and administration situation considerations among other considerations.

The frequency of dosing may be daily or once, twice, three times or more per week or per month, as needed as to effectively treat the symptoms. The timing of administration of the treatment relative to the disease itself and duration of treatment will be determined by the circumstances surrounding the case. Treatment could begin immediately, such as at the site of the injury as administered by emergency medical personnel. Treatment could begin in a hospital or clinic itself, or at a later time after discharge from the hospital or after being seen in an outpatient clinic. Duration of treatment could range from a single dose administered on a one-time basis to a life-long course of therapeutic treatments.

Although the foregoing methods appear the most convenient and most appropriate and effective for administration of proteins such as antibodies, by suitable adaptation, other effective techniques for administration, such as intraventricular administration, transdermal administration and oral administration may be employed provided proper formulation is utilized herein.

In addition, it may be desirable to employ controlled release formulations using biodegradable films and matrices, or osmotic mini-pumps, or delivery systems based on dextran beads, alginate, or collagen.

Typical dosage levels can be determined and optimized using standard clinical techniques and will be dependent on the mode of administration.

### III. Kits

A further aspect of the present disclosure provides kits comprising one or more anti-CHKV antibodies detailed above in Section I, for use in the prevention or treatment of CHIKV. The one or more anti-CHKV antibodies can have specificity to CHIKV E1 or E2 protein.

The kits provided herein generally include instructions for carrying out the methods detailed below. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

A “genetically modified” cell refers to a cell in which the nuclear, organellar or extrachromosomal nucleic acid sequences of a cell has been modified, i.e., the cell contains at least one nucleic acid sequence that has been engineered to contain an insertion of at least one nucleotide, a deletion of at least one nucleotide, and/or a substitution of at least one nucleotide.

The terms “genome modification” and “genome editing” refer to processes by which a specific nucleic acid sequence in a genome is changed such that the nucleic acid sequence is modified. The nucleic acid sequence may be modified to comprise an insertion of at least one nucleotide, a deletion of at least one nucleotide, and/or a substitution of at least one nucleotide. The modified nucleic acid sequence is inactivated such that no product is made. Alternatively, the nucleic acid sequence may be modified such that an altered product is made.

The term “heterologous” refers to an entity that is not native to the cell or species of interest.

The terms “nucleic acid” and “polynucleotide” refer to a deoxyribonucleotide or ribonucleotide polymer, in linear or circular conformation. For the purposes of the present disclosure, these terms are not to be construed as limiting with respect to the length of a polymer. The terms may encompass known analogs of natural nucleotides, as well as nucleotides that are modified in the base, sugar and/or phosphate moieties. In general, an analog of a particular nucleotide has the same base-pairing specificity, i.e., an analog of A will base-pair with T. The nucleotides of a nucleic acid or polynucleotide may be linked by phosphodiester, phosphothioate, phosphoramidite, phosphorodiamidate bonds, or combinations thereof.

The term “nucleotide” refers to deoxyribonucleotides or ribonucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “polypeptide” and “protein” are used interchangeably to refer to a polymer of amino acid residues.

As used herein, the terms “target site”, “target sequence”, or “nucleic acid locus” refer to a nucleic acid sequence that defines a portion of a nucleic acid sequence to be modified or edited and to which a homologous recombination composition is engineered to target.

The terms “upstream” and “downstream” refer to locations in a nucleic acid sequence relative to a fixed position. Upstream refers to the region that is 5′ (i.e., near the 5′ end of the strand) to the position, and downstream refers to the region that is 3′ (i.e., near the 3′ end of the strand) to the position.

Techniques for determining nucleic acid and amino acid sequence identity are known in the art. Typically, such techniques include determining the nucleotide sequence of the DNA for a gene and/or determining the amino acid sequence encoded thereby, and comparing these sequences to a second nucleotide or amino acid sequence. Genomic sequences may also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) may be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm may be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14 (6): 6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP may be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs may be found on the GenBank website. With respect to sequences described herein, the range of desired degrees of sequence identity is approximately 80% to 100% and any integer value therebetween. Typically the percent identities between sequences are at least 70-75%, 80-82%, at least 85-90%, at least 92%, at least 95%, and at least 98% sequence identity.

As used herein, the term “agronomic crop plant” refers to any crop plant grown on a production scale, most typically for the harvest of seed, silage or hay. Examples include, but are not limited to maize, soybeans, rye, wheat, oats, barley, lentils, dry peas, rape, sorghum, alfalfa, triticale, clover, and the like.

As used herein, the term “antigen” refers to any substance capable of inducing a specific immune response and of reacting with the resulting antibodies produced by that response.

As used herein, the term “antiviral” refers to a substance that interferes with the replication of a virus.

As used herein, the term “binding” refers to antigen binding affinity of an antibody, the kinetics of which are measured for example by surface plasmon resonance (SPR) and expressed by parameters of “on rate” (kₐ), “off rate” (k_{d}), and binding constant (K_{D}). “Neutralization activity” refers to the a measure of inhibitory activity of an antibody with respect to the activity of a specific virus, expressed as EC₅₀, which is the concentration of an antibody that induces half of the maximal inhibition. As used herein, “in vivo therapeutic activity” refers to the minimal concentration(s) of an antibody or its derivatives that prevent or clear infection of a virus in an animal model.

As used herein, the term “cross pollination” or “cross-breeding” when used in reference to plants means the process by which the pollen of one flower on one plant is applied (artificially or naturally) to the ovule (stigma) of a flower on another plant.

As used herein, the term “cultivar” when referring to plants means a variety, strain or race of plant that has been produced by horticultural or agronomic techniques and is not normally found in wild populations.

As used herein, the terms “dicotyledon” and “dicot” refer to a flowering plant having an embryo containing two seed halves or cotyledons. Examples include tobacco; tomato; the legumes, including peas, alfalfa, clover and soybeans; oaks; maples; roses; mints; squashes; daisies; walnuts; cacti; violets and buttercups.

As used herein, the term “plant” refers to whole plants and progeny of the whole plants, plant cells, plant tissue, plant calli, seeds and pollen. The class of plants that can be used in the methods of the disclosure is generally as broad as the class of higher plants amenable to transformation techniques, including both monocotyledonous and dicotyledonous plants.

As used herein, the term “plant line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (T0) plant regenerated from material of that line; (b) has a pedigree comprised of a T0 plant of that line; or (c) is genetically very similar due to common ancestry (e.g., via inbreeding or selfing). In this context, the term “pedigree” denotes the lineage of a plant, e.g. in terms of the sexual crosses effected such that a gene or a combination of genes, in heterozygous (hemizygous) or homozygous condition, imparts a desired trait to the plant.

As used herein, the term “endosperm” refers to a triploid structure resulting from the development of a fusion between two polar nuclei of the embryo sac and one of the sperm nucleus from the pollen found in many plant seeds. The endosperm frequently stores food materials, which are broken down during germination.

As used herein, the term “filial generation” refers to any of the generations of plant cells, tissues or organisms following a particular parental generation. The generation resulting from a mating of the parent plants is the first filial generation (designated as “F1” or “F1”), while that resulting from crossing of F1 plants is the second filial generation (designated as “F2” or “F2”).

The term “gamete” refers to a reproductive cell whose nucleus (and often cytoplasm) fuses with that of another gamete of similar origin but of opposite sex to form a zygote, which has the potential to develop into a new individual plant. Gametes are typically haploid and are differentiated into male and female.

The term “gene” refers to any segment of DNA associated with a biological function. Thus, genes include, but are not limited to, coding sequences and/or the regulatory sequences required for their expression. Genes can also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “genotype” refers to the genetic makeup of a cell, cell culture, tissue, whole organism (e.g., a whole plant or animal), or group of whole organisms (e.g., a group of plants or animals).

As used herein, the term “glycan”, which is synonymous with “polysaccharide”, refers to any linear or branched polymer consisting of monosaccharide (i.e., glucose) residues joined to each other by glycosidic linkages. Examples of glycans include glycogen, starch, hyaluronic acid, and cellulose.

As used herein, the term “glycoside” refers to any compound containing a carbohydrate molecule (sugar), particularly any such natural product in plants, convertible by hydrolytic cleavage, into a sugar and a non-sugar component.

As used herein, the term “glycopeptide” refers to a compound or composition in which carbohydrate is covalently attached to a peptide or oligopeptide.

As used herein, the term “glycoprotein” refers to a compound or composition in which carbohydrate is covalently attached to a protein.

As used herein, the term “glycosylation” refers to the addition of oligosaccharides to particular residues on a protein. This modification can be both co-translational and post-translational, occurring in the endoplasmic reticulum and golgi. Three different forms of glycosylation can be distinguished: N-linked oligosaccharides, O-linked oligosaccharides and glycosyl-phosphatidylinositol (GPI-) anchors.

As used herein, the term “hemizygous” refers to a cell, tissue or organism in which a gene is present only once in a genotype, as a gene in a haploid cell or organism, a sex-linked gene in the heterogametic sex, or a gene in a segment of chromosome in a diploid cell or organism where its partner segment has been deleted.

A “heterologous polynucleotide” or a “heterologous nucleic acid” or an “exogenous DNA segment” refer to a polynucleotide, nucleic acid or DNA segment that originates from a source foreign to the particular host cell, or, if from the same source, is modified from its original form. Thus, a heterologous gene in a host cell includes a gene that is endogenous to the particular host cell, but has been modified. Thus, the terms refer to a DNA segment that is either (i) foreign or heterologous to the cell, or (ii) homologous to the cell but in a position within the host cell nucleic acid in which the element is not ordinarily found. Exogenous DNA segments may be expressed to yield exogenous polypeptides.

A “heterologous trait” refers to a phenotype imparted to a transformed host cell or transgenic organism by an exogenous DNA segment, heterologous polynucleotide or heterologous nucleic acid.

As used herein, the term “heterozygote” refers to a diploid or polyploid individual plant cell or plant having different alleles (forms of a given gene) present at least at one locus.

As used herein, the term “heterozygous” refers to the presence of different alleles (forms of a given gene) at a particular gene locus.

As used herein, the term “HPLC” refers to High Performance Liquid Chromatography.

As used herein, the term “homozygote” refers to an individual plant cell or plant having the same alleles at one or more loci.

As used herein, the term “homozygous” refers to the presence of identical alleles at one or more loci in homologous chromosomal segments.

As used herein, the term “hybrid” refers to any cell, tissue or whole organisms (e.g., a whole plant or animal) resulting from a cross between parents that differ in one or more genes.

As used herein, the term “monoclonal antibody” or “MAb” refer to antibodies derived from a single antibody-producing cell that recognizes a specific antigen. MAbs are produced by hybridoma cells, which are a fusion of a cell that produces the antibody and a multiple myeloma cell. The myeloma cell can continuously produce the antibody.

As used herein, the term “antibody-dependent enhancement of infection (ADE)” refers to the increased risk of infection by viruses in subjects having protective immunity to another virus. Protective immunity can be due to infection by a virus or by the use of monoclonal antibody therapy against the virus.

As used herein, the term “targeted” when referring to a glycosylation profile of an antibody refers to a pre-selected glycosylation profile of the antibody.

As various changes could be made in the above-described cells and methods without departing from the scope of the invention, it is intended that all matter contained in the above description and in the examples given below, shall be interpreted as illustrative and not in a limiting sense.

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Expression and Assembly of CHKV 166 in WT and ΔXF N. Benthamiana Plants

The contribution of effector functions to mAb in vivo efficacy were evaluated by producing mAb N-glycovariants with high glycan uniformity and testing them in a mouse model of CHIKV-induced arthritis. Using a plant expression system that produces glycovariants of CHKV 152 and 166 mAbs with two distinct and homogeneous N-glycan structures, the inventors showed that the nature of antibody Fc N-glycans affects the Fc-FcγR interaction and effector functions, and in turn, modulates in vivo efficacy against CHIKV infection and foot swelling during the acute phase of disease. Although the neutralization potency of the glycovariants are identical, mAb that displayed the GnGn glycoform has higher affinity to FcγRs and superior in vivo therapeutic efficacy. In contrast, clinical protection was not as high for the glycovariant that has lower affinity to FcγRs. The mAb glycoform that conferred strong in vivo efficacy in CHIKV-challenged mice also had increased ADCC activity, suggesting that immune cells may participate in the mAb-dependent reductions in clinical diseases via ADCC when engaged by mAb glycovariants with enhanced affinity to FcγRs. The studies presented herein also provide strategies for improving the efficacy and safety of mAbs by glycan optimization using glycoengineered plants.

The DNA expression cassette of the heavy chain (HC) and light chain (LC) of CHKV 166 was cloned into a MagnICON-based plant expression vector, transformed into Agrobacterium tumefaciens, and agroinfiltrated into WT and ΔXFT N. benthamiana leaves. The expression of CHKV 166 was evaluated by Western blot analysis. As shown in FIG. 1, CHKV 166 produced in WT (WTCHKV 166) and ΔXFpCHKV plants (GnGnCHKV 166) have HC and LC with the expected molecular weight (FIG. 1A and FIG. 1B, Lanes 3-4), and are fully assembled into the heterotetramer equivalent to a positive control IgG (FIG. 1C, Lanes 3-4).

### Example 2: Purification of CHKV 166 from N. benthamiana Leaves

A two-step extraction and purification process was previously developed. The process comprises low pH precipitation and protein A chromatography for mAbs produced in plants. Using this method, CHKV 166 was purified from N. benthamiana leaves to >90% homogeneity (FIG. 2). Purified mAbs were used for further biochemical and functional analyses.

### Example 3. CHKV 152 Expression and Purification

Genes coding for CHKV 152 heavy chain (HC) and light chain (LC) were cloned into a deconstructed Geminivirus-based expression vector, transformed into Agrobacterium tumefaciens (A. tumefaciens), and transiently expressed in WT and ΔXFT N. benthamiana plant lines. Four days after agroinfiltration of gene constructs, CHKV 152 was purified from plant leaves to >90% pure with a yield of ˜ 205 g mAb per gram of leaf fresh weight (LFW).

### Example 4: N-Linked Glycosylation of Plant-Produced CHKV 152

IgGs have a conserved N-glycosylation site that plays an important structural role that affects the Fc-mediated functions of the antibody. Accordingly, the N-glycosylation of CHKV 152 and 166 produced in the WT (WTCHKV 152) and ΔXFT plants (GnGnCHKV 152) were investigated by liquid chromatography-electrospray ionization-mass spectrometry (LC-ESI-MS). As shown in Table 1, CHKV 152 produced in each type of plants carried the predominant glycoform characteristic of that plant line, with WT plants exhibiting the N-acetylglucosamine residue with xylose and α1,3 fucose (GnGnXF) while the ΔXFT plants carrying the mammalian N-glycoform of GnGn. CHKV 166 produced in ΔXFT plants exhibited the expected mammalian type GnGn N-glycan structure lacking plant-specific xylose and fucose with a high degree of glycan uniformity (>95%) (Table 1). Glycosylation pattern of 166 produced in WT plants was found to carry a mixture of N-glycans (Table 1): the major form was as the expected plant typical GnGnXF3 structure (33%), accompanied by incompletely processed complex N-glycans (39%) and oligomannosidic structures (28%).

TABLE 1
N-linked glycans of CHKV mAb variants.

| Sample | GnGnXF₃ | GnGnX | GnMXF₃ | MMXV | GnGn | Man₍₈₋₉₎ |
|---|---|---|---|---|---|---|
| GnGnCHKV 152 | | | | | >95 | |
| WTCHKV152 | 56 | 11 | 21 | 11 | | |
| GnGnCHKV 166 | | | | | >95 | |
| WTCHKV 166 | 33 | 15 | 9 | | 15 | 28 |

N-glycosylation profile as determined by liquid-chromatography electrospray ionization-mass spectrometry (LS-ESI-MS).
Numbers represent the presence of various glycoforms in percentage.
Note,
ΔXFT is a N. benthamiana N-glycosylation mutant that lacks plant specific core xylose and fucose. Glycans were annotated according to the ProGlycAn nomenclature.

### Example 5: Fc Receptor Binding of CHKV 152 Glycovariants

N-linked glycosylation in the Fc region of an antibody affects binding to Fc gamma receptors (FcγRs). The recognition and binding kinetics of WTCHKV 152 and GnGnCHKV 152 to various human FcγRs were investigated by a surface plasmon resonance (SPR) assay. GnGnCHKV 152 exhibited much stronger binding to FcγRIIIA (KD=2.996E-8 M) compared to WTCHKV 152 (KD=2.185E-7 M) (FIG. 3). The two mAb glycovariants also showed different binding kinetics to FcγRIIIA with WTCHKV 152 having faster dissociation rate than GnGnCHKV 152 (FIG. 3). WTCHKV 152 and GnGnCHKV 152 also had significant differences in FcγRI binding affinity with GnGnCHKV 152 being a more potent binder (Table 2). The two glycovariants showed similar binding affinity to FcγRIIA (Table 2).

TABLE 2
SPR binding of CHKV 152 glycovariants
to human FcγRI and FcγRIIA.

| Sample | FcγRI | FcγRIIA |
|---|---|---|
| GnGnCHKV 152 | 3.34E−9 | 2.67E−6 |
| WTCHKV152 | 1.97E−10 | 2.08E−6 |

Soluble human FcγRI or FcγRIIA was injected over GnGnCHKV 152 or WTCHKV 152 captured to the CM-5 biosensor chip. Equilibrium dissociation constant (K_{D}) was calculated by fitting and analyzed the binding data with BiAcore Evaluation Software.

### Example 6: Neutralization Activity of mAb Glycovariants

To assess the neutralization potential of the mAb glycovariants, the inhibition of CHIKV infection was measured by a focus reduction neutralization test (FRNT). Results showed that both WTCHKV 152 and GnGnCHKV 152 potently neutralized CHIKV (FIG. 4A). The potency of neutralization is almost identical between the two plant-produced glycovariants (EC50: WTCHKV 152=9.46 ng/ml and GnGnCHKV 152=11.11 ng/ml; p>0.9) and is similar to their equivalent produced in mammalian cells. Overall, plant-produced CHKV 152 glycovariants displayed similar, yet potent neutralizing activity against CHIKV.

Both WTCHKV 166 and GnGnCHKV 166 also showed strong neutralizing activity against CHIKV with statistically similar potency (GnGnCHKV 166 EC50=130.5 ng/ml vs. WTCHKV 166EC₅₀=390.8 ng/ml; p=0.33) (FIG. 4B). The mean EC50 values of plant-derived CHKV 166 are comparable to that of mammalian-cell produced orthologue (mCHKV 166 EC₅₀=154 ng/ml). Overall, plant-produced CHKV 166 glycovariants also displayed similar, yet potent neutralizing activity against CHIKV.

### Example 7: In Vivo Efficacy of CHKV 152 Glycovariants as Prophylaxis

One major hallmark of CHIKV infections is the acute viral titers and increased swelling in the joints. Among mouse models of CHIKV infection, WT mice (i.e. WT C57BL/6) develop foot/ankle swelling and arthritis symptoms similar to those observed in humans. Thus, the prophylactic efficacy of the mAbs was evaluated using the WT C57BL/6 mouse model. Five-week-old C57BL/6 mice were mock-treated or pretreated with 50 or 100 μg of WTCHKV 152, GnGnCHKV 152 or PBS intraperitoneally 24 hours before being inoculated in the footpad with 1×10⁵ PFU of CHIKV. First, the ability of mAb variants in reducing viral load in CHIKV-infected mice was examined. The viremia at 2 days post infection (dpi) was measured by RT-qPCR as viremia generally peaks at 2 dpi and subsides to undetectable levels at 4 dpi in this mouse model. Pretreatment of mice with WTCHKV 152 and GnGnCHKV 152 both markedly reduced viral loads in serum at 2 dpi relative to the PBS control (p<0.0001) (FIG. 5A). The viremia of mAb-treated mice is statistically similar to that of the mock-infected mice (p>0.99) (FIG. 5A).

Mice were also monitored for virus-induced footpad swelling daily up to 10 dpi. Consistent with their ability to reduce viremia, pretreatment of CHIKV-infected mice with as little as 50 μg of either WTCHKV 152 or GnGnCHKV 152 completely protected against CHIKV-induced swelling indistinguishable from the non-infected mice (mAb treated compared to mock-infected, p>0.9400) (FIG. 5B). In contrast, PBS-treated infected mice developed apparent swelling (mAb treated compared to PBS-treated, p<0.0004) with the typical CHIKV-induced biphasic pattern with a first (minor) peak at 2-3 dpi and the second (major) peak at 6 dpi (FIG. 5B) corresponding to tissue edema and immune cell infiltration, respectively. These data demonstrated that CHKV 152 prevented acute symptoms of CHIKV infection, therefore, supporting the prophylactic application of the mAb.

### Example 8: Therapeutic Potency of CHKV 152 Depends on its N-Glycosylation Profile

The post-exposure therapeutic efficacy of the mAb glycovariants was also evaluated by their ability in reducing viremia and foodpad swelling, as well as by histopathological analysis. Specifically, five-week-old C57BL/6 mice were inoculated in the footpad with 1×10⁵ PFU of CHIKV and then treated intraperitoneally with 50 or 100 μg of mAb glycovariants or PBS at 12 hours post infection. As observed in prophylaxis studies, WTCHKV 152 and GnGnCHKV 152 treatment both significantly reduced viremia at 2 dpi, compared to the PBS control (p<0.0001) (FIG. 6A). The serum viral loads in WTCHKV 152-treated mice were slightly lower than those of GnGnCHKV 152-treated mice, but without statistical significance (p>0.9394). Similarly, histopathological analysis of mice footpads at peak swelling (6 dpi) corroborated the therapeutic potency of the mAb glycovariants. Compared to mock-infected mice (FIG. 6B1), CHIKV-infected mice treated with PBS developed severe connective tissue damage and subcutaneous necrosis with apparent infiltration of leukocytes (FIG. 6B2). In contrast, mice treated with 100 μg of WTCHKV 152 and GnGnCHKV 152 had intact subcutaneous tissue similar to non-infected mice and much reduced connective tissue disruption (FIG. 6B 3-4). Nevertheless, leukocyte infiltration was still noticeable in the footpad of mAb treated mice.

Post-exposure treatment with 50 μg of mAb glycovariants also markedly reduced the second peak of virus-induced footpad swelling in comparison to the PBS-treat control (p<0.0001) (FIG. 7A). Notably, the results revealed significant differences in swelling depending on the glycan profile of the mAb. WTCHKV 152 reduced the footpad swelling at 5, 6, and 7 dpi (p<0.0001 compared to PBS-treated) (FIG. 7A) and the swelling was further reduced in mice treated with GnGnCHKV 152, reaching statistical significance at 6 dpi, (compared with WTCHKV 152, p<0.0015) (FIG. 7A and FIG. 7B). These data suggest that Fc effector functions contribute to the efficacy of CHKV 152 and these functions can be modulated by N-glycan variation which affects Fc and FcγR interactions.

### Example 9: Antibody Dependent Cellular Cytotoxicity of CHKV 152 Glycovariants

To define how Fc effector functions may contribute to the potency of anti-CHIKV mAbs, the natural killer (NK) cell-mediated antibody dependent cellular cytotoxicity (ADCC) activity of CHKV 152 glycovariants was examined. Uninfected or CHIKV-infected Vero cells (target cell) were incubated with PBS, WTCHKV 152, GnGnCHKV 152, or a IgG isotype negative control and then exposed to NK cells (effector cell); and target cell killing (lysis) was measured. WTCHKV 152 and GnGnCHKV 152 did not show specific ADCC activity to uninfected target cells even at high concentrations when compared to PBS or the isotype negative control (p>0.24) (FIG. 8A). In contrast, WTCHKV 152 and GnGnCHKV 152 both showed strong ADCC activity against CHIKV-infected cells at concentrations ≥1 μg/ml (p<0.0001, compared to PBS and IgG isotype negative control) (FIG. 8B). At lower concentrations, however, significant differences in ADCC activity was observed depending on then N-glycosylation of the mAb, with GnGnCHKV 152 mediating dramatic higher ADCC activity than WTCHKV 152 (p<0.0001) (FIG. 8B). The enhanced ADCC activity of GnGnCHKV 152 correlates with its increased in vivo therapeutic potency over WTCHKV 152. These results demonstrated that CHKV 152 are able to activated immune cells to clear CHIKV-infected cells via ADCC, which may serve as one of the mechanisms of Fc effectors functions for the observed enhancement of in vivo efficacy by GnGnCHKV 152.

### Discussion for Examples 1-9

Here, the contribution of effector functions to mAb in vivo efficacy was evaluated by producing mAb N-glycovariants with high glycan uniformity and testing them in a mouse model of CHIKV-induced arthritis. Using a plant expression system that produces glycovariants of anti-CHIKV E1 and E2 mAbs with two distinct and homogeneous N-glycan structures, it is shown that the nature of antibody Fc N-glycans affects the Fc-FcγR interaction and effector functions, and in turn, modulates in vivo efficacy against CHIKV infection and foot swelling during the acute phase of disease. Although the neutralization potency of the glycovariants are identical, the mAb that displayed the GnGn glycoform has higher affinity to FcγRs and superior in vivo therapeutic efficacy. In contrast, clinical protection was not as high for the glycovariant that has lower affinity to FcγRs. The mAb glycoform that conferred strong in vivo efficacy in CHIKV-challenged mice also had increased ADCC activity, indicating that immune cells may participate in the mAb-dependent reductions in clinical diseases via ADCC when engaged by mAb glycovariants with enhanced affinity to FcγRs.

For example, mAbs against the preS1 domain of Hepatitis B virus (HBV) large envelope protein protect mice from HBV infection largely through Fc-mediated immune effector functions. FcγR interactions were also shown to contribute to the therapeutic activity of broadly neutralizing mAbs against human immunodeficiency virus (HIV). The impact of Fc effector function for CHIKV mAb efficacy has only began to emerge very recently. Using transgenic mice that lack activating FcγRs or mAb mutants that completely forgo FcγR engagement, Fc-FcγR interactions were found to be required for the full therapeutic potency of anti-CHIV E1 and E2 human IgG1-type mAbs in a WT mouse mode.

The present disclosure addresses the participation of antibody Fc effector functions in the overall efficacy of mAb-based therapy for viral infections. The studies presented herein use glycovariants of an anti-CHIKV E2 and E1 human IgG1 mAbs that have either baseline or enhanced FcγR engagement activities, respectively. The results confirm the contribution of Fc effector functions in reducing clinical diseases, but also for the first time clarify how mAb sugar moieties regulate Fc effector functions and in vivo efficacy against CHIKV.

The interaction between the IgG Fc domain and FcγR on immune cells is highly sensitive to the composition of N-linked Fc glycans. However, the impact of glycosylation on the overall efficacy of mAb-based drugs is not thoroughly understood. Studies on the clinical efficacy of anti-cancer mAb drugs have shown that activation of antibody effector functions is a major mechanism of action for approved drugs including rituximab, trastuzumab and cetuximab, which have been shown to kill cancer cells by their potent ADCC or CDC activities. However, studying the influence of sugars on effector functions and improving effector function by glycan manipulation are often impeded by the difficulty of producing mAbs with homogenous N-glycans. MAbs produced in mammalian cells usually carry a mixture of N-glycans, with some glycoforms being more bioactive than others. In contrast, plant-produced mAbs usually bear a single dominant N-glycan structure due to a drastically reduced repertoire of glycoenzymes in plant cells. Consequently, a portfolio of N. benthamiana lines have been developed by glycoengineering to produce mAbs that carry various distinct yet uniform mammalian glycoforms, offering the opportunity of producing mAbs with tailor-made N-glycans. In this study, a GnGn mAb glycovariant was generated using a N. benthamiana mutant lacking core xylose and fucose. This GnGn glycovariant exhibited increased binding to FcγRs, enhanced ADCC activity, and more potent in vivo efficacy against CHIKV infection over its WT counterpart, revealing the importance of N-glycan structures, especially the GnGn structure, for the Fc effector functions and in vivo efficacy of CHIKV mAbs. Since the GnGn glycoform does not affect the antigen binding and the serum half-life of IgGs, the strategy of using glycoengineered plants to optimize mAbs may also be applicable to other mAbs.

NK cells, neutrophils and macrophages are examples of immune cells that express activating FcγRs that can be engaged by the Fc region of mAbs to clear infected cells via ADCC or antibody-dependent cell-mediated phagocytosis (ADCP). NK cells are an important mediator of innate immune defense and their importance in controlling early viral infections have been demonstrated for several human viruses including HIV, Herpes simplex virus (HSV), and Cytomegalovirus (CMV). The results presented herein demonstrate that an anti-CHIKV E2 mAb with homogenous GnGn N-glycans are able to activate human NK cells to clear CHIKV-infected cells via ADCC, suggesting a potential role of NK cells in fighting human CHIKV infection. FcγRIIIA usually has low affinities to IgGs. Due to its increased affinity to FcγRIIIA, glycovariant GnGnCHKV 152 may be more potent in activating NK-mediated ADCC than regular human IgGs with baseline affinities. The mouse ortholog of human FcγRIIIA (mFcγRIV) is not expressed on mouse NK cells but on mouse monocytes and neutrophils. Since CHKV 152 is a human IgG1 which has similar Fc and complement binding specificities as mouse IgG2a/c to mouse FcγRs), GnGnCHKV 152 may also have enhanced binding to mFcγRIV, leading to increased activation of ADCC via mouse monocytes and neutrophils, and in turn, the observed enhancement of in vivo efficacy. As GnGnCHKV 152 also displayed enhanced binding to other FcγRs, other immune cells expressing these FcγRs and complement may also contribute to the enhanced in vivo efficacy. Further studies of mAb glycovariants with enhanced affinity to FcγRs are warranted to understand the effect of Fc-FcγR affinity on the engagement of various immune cells in viral clearance and reduction of inflammation/arthritis during acute CHIKV infection.

The results presented herein also indicate that anti-CHIKV human mAbs can be effective prophylaxis to prevent infection during epidemics, especially for populations with high risk of developing severe diseases. Combined with the rapid and low cost nature of plant transient expression systems, the study also demonstrates that glycoengineered plants provide a powerful strategy for the rapid screening and optimization of mAb therapeutics.

### Materials and Methods for Examples 1-9

### Vector Construction for Expression of CHKV 152 and 166

The coding sequence of CHKV 152 and 166 variable region of HC (VH) and LC (VL) was fused to the corresponding DNA sequences of human IgG1 constant regions of HC (CH) and LC (CL), respectively. The resulting HC and LC coding sequences were cloned into a Geminiviral vector (pBYR11eK2Md) or MagnICON-based plant expression vectors pICH21595 and pICH11599, and transformed into A. tumefaciens as described previously (FIG. 9).

Transient Expression of CHKV 152 and 166 in N. benthamiana Leaves

Wild-type and ΔXFT N. benthamiana plants were grown and agroinfiltrated with A. tumefaciens strains that contain CHKV152 or 166 HC and LC constructs.

Extraction and Purification of pCHKV 152 and 166 from Plants

Agroinfiltrated leaves were harvested 4 dpi (CHKV 152) or 7 dpi (CHKV 166) and mAbs were extracted and purified. Briefly, the crude leaf extract was obtained by homogenization in extraction buffer (1×PBS pH5.2, 10 mg/ml Sodium Ascorbate, 1 mM EDTA) and clarified by centrifugation at 15,000×g for 30 min at 4° C. CHKV 152 in clarified protein extract were purified by a two-step purification process comprised of low pH precipitation followed by protein A affinity chromatography.

### Protein Analyses

SDS-PAGE (10% or 4-20% gradient) electrophoresis was performed either under reducing (5% v/v β-mercaptoethanol) or non-reducing conditions. SDS-PAGE gels were stained with Coomassie blue. For western blot analysis, proteins on SDS-PAGE gels were transferred onto PVDF membranes and detected with horseradish peroxidase (HRP)-conjugated antibodies against human-kappa LC or gamma HC (Southern Biotech).

The expression level of CHKV 152 and 166 was measured by an ELISA that detected the fully assembled form of mAbs with both HC and LC. Briefly, plates were coated with an anti-human gamma HC antibody (Southern Biotech) and incubated with the plant protein extract. After washing, a HRP-conjugated anti-human-kappa LC antibody (Southern Biotech) was used for detection. A plant produced mAb with human IgG1 CH and kappa CL (E16) was used as a reference standard.

### N-Linked Glycan Analysis

LC-ESI-MS was used to determine the N-linked glycosylation profiles of CHKV 152 or 166 variants as previously reported. Briefly, the HC band of purified CHKV mAbs on Coomassie-stained SDS-PAGE was excised from the gel. Peptide fragments were generated by S-alkylation and tryptic or tryptic/GluC digestion and subsequently eluted from the gel with 50% acetonitrile. Peptide fragments were then separated on a Reversed Phase Column (150×0.32 mm BioBasic-18, Thermo Fisher Scientific) with a 1%-80% acetonitrile gradient. Glycopeptides were analyzed with a quadruple time-of-flight (Q-TOF) Ultima Global mass spectrometer (Waters, Milford, MA, USA). Spectra were summed and deconvoluted for identification of glycoforms. The ProGlycAn nomenclature (www.proglycan.com) was used to annotate the glycans.

### Virus and Cells

CHIKV (strain LR-OPY1) was propagated and tittered in Vero cells (ATCC, CCL-81). Vero cells were cultured in DMEM supplemented with 10% FBS at 37° C. in an incubation with 5% CO2.

### CHIKV Neutralization and Plaque Assay

Plaque reduction neutralization test (PRNT) was performed as previously described. Specifically, mAbs were serially diluted in phosphate-buffered saline (PBS), while CHIKV was diluted in serum-free DMEM to a working concentration of 100 plaque forming units (PFU) per well. Following dilutions, mAbs were added to CHIKV and incubated for 1 hr at 37° C. CHIKV-mAb mixes were then added to a 90-95% confluence Vero cells in a 6-well tissue culture plate and incubated for 1 hr at 37° C. for virus attachment. After removing unattached virus-mAbs in the medium, cells were overlaid with fresh media (complete DMEM containing 1% sea plaque agarose, Lonza) and incubated for an additional 3 days at 37° C. Plaques were visualized by staining with neutral red (Sigma) and counted. Percent (%) neutralization was calculated as: [(number of CHIKV plaque per well with no mAb)-(number of CHIKV plaque per well of diluted mAb)/(number of CHIKV plaque per well with no mAb)×100]. Experiments were repeated twice. The half maximal effective concentration (EC50) of each mAb was calculated using GraphPad Prism software (Version 6.0).

### Animal Studies

Female C57BL/6J mice (5 weeks old, 6 mice per group) were purchased from the Jackson Laboratory and subcutaneously inoculated on the ventral side of the right hind footpad with 105 PFUs of CHIKV. Mice were intraperitoneally inoculated with anti-CHIKV MAbs before (prophylactic treatment) or after (post-exposure therapeutic treatment) CHIKV infection. The height (thickness) and breath (width) of the peri-metatarsal area of inoculated footpad were measured daily for up to 10-day post infection (dpi.) by using a digital caliper (Electron Microscopy Science) and the footpad swelling was expressed as the relative increase in footpad compared to pre-infection. In addition, mice were bled retro-orbitally at 2 dpi. to measure viremia.

### Real Time-Quantitative PCR (RT-qPCR)

Total RNA was isolated from mouse blood samples using TRI-reagent (Molecular Research Center, Inc.). The first-strand complementary DNA (cDNA) was synthesized by using the iSCRIPT cDNA synthesis kit (Bio-Rad). CHIKV envelope protein 1 (CHIKV E1) and cellular β-actin RNA copy numbers were determined by RT-qPCR in a CFX96 Real-Time system (Bio-Rad) using SYBR Green supermix (Bio-Rad). Viral copy numbers were expressed as the ratio of CHIKV E1 to cellular β-actin.

### Histology

Histological examination of mice footpad was done as previously described. Briefly, representative CHIKV-infected mice were sacrificed and footpads were collected at peak swelling (6 dpi). After an overnight fixation in 4% paraformaldehyde followed by decalcification in 10% EDTA for over 10 days, tissues were paraffin embedded and sectioned (10 μm) with a microtome (American Optical Spencer 820). Hematoxylin and eosin (H&E) stained images were acquired in a bright-field microscope (Olympus BH2).

### ADCC Assay for CHIKV Infected and Non-Infected VERO Cells

Culture dishes (100 mm) were seeded with 3×10⁶ Vero cells in DMEM with 5% FBS. Twenty-four hours later, cells were mock-infected or infected with 0.05 MOI of CHIKV and incubated for additional 48 hours. Vero cells were then rinsed with PBS and dislodged using a cell scrapper in PBS with 8 mM EDTA, and resuspended in MEM media with 5% FBS at 1 million/ml. Subsequently, Calcein AM (Invitrogen, C3099) was added to Vero cells to a final concentration of 5 μg/ml. After 1-hour incubation at 37° C., Vero cells were washed and resuspended in RPMI with 10% FBS at 0.1 million/ml. Vero cells were incubated with mAb variants at room temperature for 15 minutes before the initiation of the assay.

In the meantime, NK cells were expanded from human peripheral blood mononuclear cells (PBMCs) and cultured in RPMI with 10% FBS plus 50 unit/ml human IL-2 for 24 hours. To initiate the assay, IL-2 treated NK cells and mAb-Vero cell mixture were added to wells of a V bottom plate at a density for each cell population to achieve a Effector cell/Target cell (E/T) ratio of 1.25 to 1. The plate was incubated at 37° C. for 4 hours before centrifuged at 100×g for 5 minutes. The supernatant which contained the Calcein AM released by lysed target cells was transferred to a black assay plate with clear bottom for fluorescence reading (excitation=485 nm, emission=530 nm). For control wells, Triton X-100 (2%) or RPMI media was used in place of NK cells to measure “maximum release” and “spontaneous release”, respectively. The % of target cell lysis was calculated according to the formula=[(test release-spontaneous release)/(maximum release-spontaneous release)]×100. Each sample was measured in triplicates.

### Example 10. Antibody-Dependent Enhancement Activity of Plant-Produced CHKVmab for DENV Infection

One of the challenges for mAb therapeutics against viral infections is the increased risk of infection by other viruses in treated patients via the mechanism of antibody-dependent enhancement of infection (ADE). For example, cross-reactive but sub-neutralizing antibodies against one serotype of DENV from a previous infection can form complexes with another serotype of DENV during a secondary infection to promote viral infection of FccR-bearing myeloid cells, predisposing patients to develop the more severe dengue haemorrhagic fever/dengue shock syndrome through ADE. Since the E proteins of CHIKV and DENV are related and the two viruses cocirculate geographically, It was examined whether CHKVmab would enhance the infection of DENV.

The potential enhancing activities of the anti-CHIKV mAbs for DENV infection were examined by using FccRIIa+ K562 cells (ATCC, CCL-2243). Serial dilutions of CHKVmabs or the positive control mAb 4G2 were initially incubated with DENV-2 (ATCC, VR-1584) for 1 hr at 37° C. Thereafter, the mAb-virus complexes were incubated with K562 cells (MOI=1) for 48 h. Cells were then fixed with 4% paraformaldehyde (Sigma), permeabilized with 0.1% saponin (Sigma), and stained with Alexa 488 (Invitrogen)-conjugated 4G2 (ATCC, HB112). Stained cells were washed and analysed with a Navios flow cytometer (Beckman Coulter) to determine the percentage of infected cells.

As previously demonstrated, the 4G2, a mAb that cross-reacts with E of most flaviviruses used here as a positive control, efficiently caused ADE of DENV infection in K562 cells that express the human FccR (FIG. 10). In contrast, ΔXFpCHKVmab, used here as a negative control, and WTpCHKVmab did not promote ADE for DENV (FIG. 10). These results indicate that plant-produced CHKVmabs are not only efficacious but also safe as potential treatment against CHIKV infection.

SEQUENCE TABLE

| SEQ | | |
|---|---|---|
| ID | | |
| NO | Sequence | Name |
| 1 | CGATCGGTCGATTCATAGAAGATTAGATTT | pBYR11eK2Md |
| | TTCATAGTATTTTTTTAAAGTAAACCTTTA | plasmid |
| | ACTACGGTTAGGACACTTTTAAGTTAAATT | |
| | TAATTTGAACCCTTAAATTAATTTTTAAAA | |
| | TAGATAAATATCAATCATCCTGATATGCTT | |
| | TTGAAAAAATGAATGAGAAAGATGATTCAA | |
| | TTAAGGCCACATTTTAATCATGACTAAAAT | |
| | AATATACAGTATAATTTCATATATATTTGC | |
| | TTTAAAAAAAAATTGACAATCCATTCGTTT | |
| | CTAGCAATAAATTTCTTCAACCACAAATAT | |
| | ATTAAAGATAACTACGGCATAGAAACAAAA | |
| | ATCTATGAAGAATTTTTGTATACTTCATAT | |
| | GAAATTAAAAAAAACTTCATTGAACATCAA | |
| | AATAATAATAATAATCATAAACTCCTCAAT | |
| | ATTTATATTCCTAGCTTCTTGAATTAAATT | |
| | GTTTACATATTCAACGATGTAAAAAATTAT | |
| | TTCTCTATCTATTTTCCTTATATCATGCAT | |
| | GGTTTCACATATATCAAAGGATAAAAGCAA | |
| | TCTATGTAAATTATCTCACTTTATTAAGTT | |
| | TTCTATCTGAATTATTGAGAACGTAGATTT | |
| | CTTTTTGCACTATCCCCCAATAATTAGCAA | |
| | AACACACCTAGACTAGATTTGTTTTGCTAA | |
| | CCCAATTGATATTAATTATATATGATTAAT | |
| | ATTTATATGTATATGGAATTGGTTAATAAA | |
| | ATGCATCTGGTTCATCAAAGAATTATAAAG | |
| | ACACGTGACATTCATTTAGGATAAGAAATA | |
| | TGGATGATCTCTTTCTCTTATTCAGATAAT | |
| | TAGTAATTACACATAACACACAACTTTGAT | |
| | GCCCACATTATAGTGATTAGCATGTCACTA | |
| | TGTGTGCATCCTTTTATTTCATACATTAAT | |
| | TAACTTGGCCAATCCAGAAGATGGACAAGT | |
| | CTAGGGTCACATTGCAGGGTACTCTAGcTT | |
| | ACTCGCCTTCTTTTTCGAAGGTTTGAGTAC | |
| | CTTCAGGGCATCCTCTTGATACATTACTTT | |
| | CCACTTCGATTGGGGCAAGCTGTAGCAGTT | |
| | CTTGCTTAGACCGAATTGCCATCTCACAGA | |
| | GATGCTGAAGAGTTCGCGACCCTCCAGAAA | |
| | CGGTGATACTAACTCCTCGAAACCGAATAC | |
| | TATAGGTACATCCGATCTGGTCGAAACCGA | |
| | AAAATCGAGATGCTGCATAGTTAACCGAAT | |
| | CTCCCGTCCAAGATCCAAGGACTCTGTGCA | |
| | GTGAAGCTTCCGTCCTGTCGTATCTGAGAT | |
| | ATCTCTTAAATACAACTTTCCCGAAACCCC | |
| | AGCTTTCCTTGAAACCAAGGGGATTATCTT | |
| | GATTCGAATTCGTCTCATCGTTATGTAGCC | |
| | GCCACTCAGTCCAACTCGGACTTTCGTCAG | |
| | GAAGTTTGAAGGGAGAAGTtGTACCTCCTG | |
| | ATCCTCCATCCCAACGTTCACTGTTAGCTT | |
| | GTTCCCTAGCGTCGTTTCCTTGTATAGCTC | |
| | GTTCCATGGATTGTAAATAGTAATTGTAAT | |
| | GTTGTTTGTTGTTTGTTGTTGTTGGTAATT | |
| | GTTGTAAAAATACGCTCTCCAAATGAAATG | |
| | AACTTCCTTATATAGAGGAAGGGTCTTGCG | |
| | AAGGATAGTGGGATTGTGCGTCATCCCTTA | |
| | CGTCAGTGGAGATATCACATCAATCCACTT | |
| | GCTTTGAAGACGTGGTTGGAACGTCTTCTT | |
| | TTTCCACGATGCTCCTCGTGGGTGGGGGTC | |
| | CATCTTTGGGACCACTGTCGGCAGAGGCAT | |
| | CTTCAACGATGGCCTTTCCTTTATCGCAAT | |
| | GATGGCATTTGTAGGAGCCACCTTCCTTTT | |
| | CCACTATCTTCACAATAAAGTGACAGATAG | |
| | CTGGGCAATGGAATCCGAGGAGGTTTCCGG | |
| | ATATTACCCTTTGTTGAAAAGTCTCAATTG | |
| | CCCTTTGGTCTTCTGAGACTGTATCTTTGA | |
| | TATTTTTGGAGTAGACAAGTGTGTCGTGCT | |
| | CCACCATGTTCTGGCAATTCCGGTTCGCTT | |
| | GCTGTCCATAAAACCGCCCAGTCTAGCTAT | |
| | CGCCATGTAAGCCCACTGCAAGCTACCTGC | |
| | TTTCTCTTTGCGCTTGCGTTTTCCCTTGTC | |
| | CAGATAGCCCAGTAGCTGACATTCATCCGG | |
| | GGTCAGCACCGTTTCTGCGGACTGGCTTTC | |
| | TACGTGTTCCGCTTCCTTTAGCAGCCCTTG | |
| | CGCCCTGAGTGCTTGCGGCAGCGTGAagct | |
| | ggcgcgCCGCTCTAGCAGAAGGCATGTTGT | |
| | TGTGACTCCGAGGGGTTGCCTCAAACTCTA | |
| | TCTTATAACCGGCGTGGAGGCATGGAGGCA | |
| | AGGGCATTTTGGTAATTTAAGTAGTTAGTG | |
| | GAAAATGACGTCATTTACTTAAAGACGAAG | |
| | TCTTGCGACAAGGGGGGCCCACGCCGAATT | |
| | TTAATATTACCGGCGTGGCCCCACCTTATC | |
| | GCGAGTGCTTTAGCACGAGCGGTCCAGATT | |
| | TAAAGTAGAAAAGTTCCCGCCCACTAGGGT | |
| | TAAAGGTGTTCACACTATAAAAGCATATAC | |
| | GATGTGATGGTATTTGATGGAGCGTATATT | |
| | GTATCAGGTATTTCCGTCGGATACGAATTA | |
| | TTCGTACGACCCTCCTGCAGGTCAACATGG | |
| | TGGAGCACGACACACTTGTCTACTCCAAAA | |
| | ATATCAAAGATACAGTCTCAGAAGACCAAA | |
| | GGGCAATTGAGACTTTTCAACAAAGGGTAA | |
| | TATCCGGAAACCTCCTCGGATTCCATTGCC | |
| | CAGCTATCTGTCACTTTATTGTGAAGATAG | |
| | TGGAAAAGGAAGGTGGCTCCTACAAATGCC | |
| | ATCATTGCGATAAAGGAAAGGCCATCGTTG | |
| | AAGATGCCTCTGCCGACAGTGGTCCCAAAG | |
| | ATGGACCCCCACCCACGAGGAGCATCGTGG | |
| | AAAAAGAAGACGTTCCAACCACGTCTTCAA | |
| | AGCAAGTGGATTGATGTGATAACATGGTGG | |
| | AGCACGACACACTTGTCTACTCCAAAAATA | |
| | TCAAAGATACAGTCTCAGAAGACCAAAGGG | |
| | CAATTGAGACTTTTCAACAAAGGGTAATAT | |
| | CCGGAAACCTCCTCGGATTCCATTGCCCAG | |
| | CTATCTGTCACTTTATTGTGAAGATAGTGG | |
| | AAAAGGAAGGTGGCTCCTACAAATGCCATC | |
| | ATTGCGATAAAGGAAAGGCCATCGTTGAAG | |
| | ATGCCTCTGCCGACAGTGGTCCCAAAGATG | |
| | GACCCCCACCCACGAGGAGCATCGTGGAAA | |
| | AAGAAGACGTTCCAACCACGTCTTCAAAGC | |
| | AAGTGGATTGATGTGATATCTCCACTGACG | |
| | TAAGGGATGACGCACAATCCCACTATCCTT | |
| | CGCAAGACCCTTCCTCTATATAAGGAAGTT | |
| | CATTTCATTTGGAGAGGACCTCGAGAAACA | |
| | AACAAAATCAACAAATATAGAAAATAACGC | |
| | ATTTCCAATTCTTTGAAATTTCTGCAACAT | |
| | ctagcgagaccaacaacggtctctagctcg | |
| | aagtgacatcacaaagttgaaggtaataaa | |
| | gccaaattaattaagacattttcataatga | |
| | tgtcaagaatgcaaagcaaattgcataact | |
| | gcctttatgcaaaacattaatataatataa | |
| | attataaagaactgcgctctctgcttctta | |
| | ttttcttagcttcatttattagtcactagc | |
| | tgttcagaattttcagtatcttttgatatt | |
| | actaagaacctaatcacacaatgtatattc | |
| | ttatgcaggaaaagcagaatgctgagctaa | |
| | aagaaaggctttttccattttcgagagaca | |
| | atgagaaaagaagaagaagaagaagaagaa | |
| | gaagaagaagaaaagagtaaataataaagc | |
| | cccacaggaggcgaagttcttgtagctcca | |
| | tgttatctaagttattgatattgtttgccc | |
| | tatattttatttctgtcattgtgtatgttt | |
| | tgttcagtttcgatctccttgcaaaatgca | |
| | gagattatgagatgaataaactaagttata | |
| | ttattatacgtgttaatattctcctcctct | |
| | ctctagctagccttttgttttctctttttc | |
| | ttatttgattttctttaaatcaatccattt | |
| | taggagagggccagggagtgatccagcaaa | |
| | acatgaagattagaagaaacttccctcttt | |
| | tttttcctgaaaacaatttaacgtcgagat | |
| | ttatctctttttgtaatggaatcatttcta | |
| | cagttatgacgaattgtacatcaacgaaaa | |
| | attagtcaaacgactaaaataaataaatat | |
| | catgtgttattaagaaaattctcctataag | |
| | aatattttaatagatcatatgtttgtaaaa | |
| | aaaattaatttttactaacacatatattta | |
| | cttatcaaaaatttgacaaagtaagattaa | |
| | aataatattcatctaacaaaaaaaaaacca | |
| | gaaaatgctgaaaacccggcaaaaccgaac | |
| | caatccaaaccgatatagttggtttggttt | |
| | gattttgatataaaccgaaccaactcggtc | |
| | catttgcacccctaatcataatagctttaa | |
| | tatttcaagatattattaagttaacgttgt | |
| | caatatcctggaaattttgcaaaatgaatc | |
| | aagcctatatggctgtaatatgaatttaaa | |
| | agcagctcgatgtggtggtaatatgtaatt | |
| | tacttgattctaaaaaaatatcccaagtat | |
| | taataatttctgctaggaagaaggttagct | |
| | acgatttacagcaaagccagaatacaaaga | |
| | accataaagtgattgaagctcgaaatatac | |
| | gaaggaacaaatatttttaaaaaaatacgc | |
| | aatgacttggaacaaaagaaagtgatatat | |
| | tttttgttcttaaacaagcatcccctctaa | |
| | agaatggcagttttcctttgcatgtaacta | |
| | ttatgctcccttcgttacaaaaattttgga | |
| | ctactattgggaacttcttctgaaaatagt | |
| | ggtaccGAGTGTACTTCAAGTCAGTTGGAA | |
| | ATCAATAAAATGATTATTTTATGAATATAT | |
| | TTCATTGTGCAAGTAGATAGAAATTACATA | |
| | TGTTACATAACACACGAAATAAACAAAAAA | |
| | ACACAATCCAAAACAAACACCCCAAACAAA | |
| | ATAACACTATATATATCCTCGTATGAGGAG | |
| | AGGCACGTTCAGTGACTCGACGATTCCCGA | |
| | GCAAAAAAAGTCTCCCCGTCACACATATAG | |
| | TGGGTGACGCAATTATCTTCAAAGTAATCC | |
| | TTCTGTTGACTTGTCATTGATAACATCCAG | |
| | TCTTCGTCAGGATTCCAAAGAATTATAGAA | |
| | GGGATCGGTCAACATGGTGGAGCACGACAC | |
| | ACTTGTCTACTCCAAAAATATCAAAGATAC | |
| | AGTCTCAGAAGACCAAAGGGCAATTGAGAC | |
| | TTTTCAACAAAGGGTAATATCCGGAAACCT | |
| | CCTCGGATTCCATTGCCCAGCTATCTGTCA | |
| | CTTTATTGTGAAGATAGTGGAAAAGGAAGG | |
| | TGGCTCCTACAAATGCCATCATTGCGATAA | |
| | AGGAAAGGCCATCGTTGAAGATGCCTCTGC | |
| | CGACAGTGGTCCCAAAGATGGACCCCCACC | |
| | CACGAGGAGCATCGTGGAAAAAGAAGACGT | |
| | TCCAACCACGTCTTCAAAGCAAGTGGATTG | |
| | ATGTGATAACATGGTGGAGCACGACACACT | |
| | TGTCTACTCCAAAAATATCAAAGATACAGT | |
| | CTCAGAAGACCAAAGGGCAATTGAGACTTT | |
| | TCAACAAAGGGTAATATCCGGAAACCTCCT | |
| | CGGATTCCATTGCCCAGCTATCTGTCACTT | |
| | TATTGTGAAGATAGTGGAAAAGGAAGGTGG | |
| | CTCCTACAAATGCCATCATTGCGATAAAGG | |
| | AAAGGCCATCGTTGAAGATGCCTCTGCCGA | |
| | CAGTGGTCCCAAAGATGGACCCCCACCCAC | |
| | GAGGAGCATCGTGGAAAAAGAAGACGTTCC | |
| | AACCACGTCTTCAAAGCAAGTGGATTGATG | |
| | TGATATCTCCACTGACGTAAGGGATGACGC | |
| | ACAATCCCACTATCCTTCGCAAGACCCTTC | |
| | CTCTATATAAGGAAGTTCATTTCATTTGGA | |
| | GAGGACCTCGAGAAACAAACAAAATCAACA | |
| | AATATAGAAAATAACGCATTTCCAATTCTT | |
| | TGAAATTTCTGCAACATCTAGAGGATCCCC | |
| | GGGTACCGAGCTcgaagtgacatcacaaag | |
| | ttgaaggtaataaagccaaattaattaaga | |
| | cattttcataatgatgtcaagaatgcaaag | |
| | caaattgcataactgcctttatgcaaaaca | |
| | ttaatataatataaattataaagaactgcg | |
| | ctctctgcttcttattttcttagcttcatt | |
| | tattagtcactagctgttcagaattttcag | |
| | tatcttttgatattactaagaacctaatca | |
| | cacaatgtatattcttatgcaggaaaagca | |
| | gaatgctgagctaaaagaaaggctttttcc | |
| | attttcgagagacaatgagaaaagaagaag | |
| | aagaagaagaagaagaagaagaagaaaaga | |
| | gtaaataataaagccccacaggaggcgaag | |
| | ttcttgtagctccatgttatctaagttatt | |
| | gatattgtttgccctatattttatttctgt | |
| | cattgtgtatgttttgttcagtttcgatct | |
| | ccttgcaaaatgcagagattatgagatgaa | |
| | taaactaagttatattattatacgtgttaa | |
| | tattctcctcctctctctagctagcctttt | |
| | gttttctctttttcttatttgattttcttt | |
| | aaatcaatccattttaggagagggccaggg | |
| | agtgatccagcaaaacatgaagattagaag | |
| | aaacttccctcttttttttcctgaaaacaa | |
| | tttaacgtcgagatttatctctttttgtaa | |
| | tggaatcatttctacagttatgacgaattg | |
| | tacatcaacgaaaaattagtcaaacgacta | |
| | aaataaataaatatcatgtgttattaagaa | |
| | aattctcctataagaatattttaatagatc | |
| | atatgtttgtaaaaaaaattaatttttact | |
| | aacacatatatttacttatcaaaaatttga | |
| | caaagtaagattaaaataatattcatctaa | |
| | caaaaaaaaaaccagaaaatgctgaaaacc | |
| | cggcaaaaccgaaccaatccaaaccgatat | |
| | agttggtttggtttgattttgatataaacc | |
| | gaaccaactcggtccatttgcacccctaat | |
| | cataatagctttaatatttcaagatattat | |
| | taagttaacgttgtcaatatcctggaaatt | |
| | ttgcaaaatgaatcaagcctatatggctgt | |
| | aatatgaatttaaaagcagctcgatgtggt | |
| | ggtaatatgtaatttacttgattctaaaaa | |
| | aatatcccaagtattaataatttctgctag | |
| | gaagaaggttagctacgatttacagcaaag | |
| | ccagaatacaaagaaccataaagtgattga | |
| | agctcgaaatatacgaaggaacaaatattt | |
| | ttaaaaaaatacgcaatgacttggaacaaa | |
| | agaaagtgatatattttttgttcttaaaca | |
| | agcatcccctctaaagaatggcagttttcc | |
| | tttgcatgtaactattatgctcccttcgtt | |
| | acaaaaattttggactactattgggaactt | |
| | cttctgaaaatagtggtaccGAGTGTACTT | |
| | CAAGTCAGTTGGAAATCAATAAAATGATTA | |
| | TTTTATGAATATATTTCATTGTGCAAGTAG | |
| | ATAGAAATTACATATGTTACATAACACACG | |
| | AAATAAACAAAAAAACACAATCCAAAACAA | |
| | ACACCCCAAACAAAATAACACTATATATAT | |
| | CCTCGTATGAGGAGAGGCACGTTCAGTGAC | |
| | TCGACGATTCCCGAGCAAAAAAAGTCTCCC | |
| | CGTCACACATATAGTGGGTGACGCAATTAT | |
| | CTTCAAAGTAATCCTTCTGTTGACTTGTCA | |
| | TTGATAACATCCAGTCTTCGTCAGGATTgC | |
| | AAAGAATTATAGAAGGGATCCCACCTTTTA | |
| | TTTTCTTCTTTTTTCCATATTTAGGGTTGA | |
| | CAGTGAAATCAGACTGGCAACCTATTAATT | |
| | GCTTCCACAATGGGACGAACTTGAAGGGGA | |
| | TGTCGTCGATGATATTATAGGTGGCGTGTT | |
| | CATCGTAGTTGGTGAAGTCGATGGTCCCGT | |
| | TCCAGTAGTTGTGTCGCCCGAGACTTCTAG | |
| | CCCAGGTGGTCTTTCCGGTACGAGTTGGTC | |
| | CGCAGATGTAGAGGCTGGGGTGTCTGACCC | |
| | CAGTCCTTCCCTCATCCTGGTTAGATCGGC | |
| | CATCCACTCAAGGTCAGATTGTGCTTGATC | |
| | GTAGGAGACAGGATGTATGAAAGTGTAGGC | |
| | ATCGATGCTTACATGATATAGGTGCGTCTC | |
| | TCTCCAGTTGTGCAGATCTTCGTGGCAGCG | |
| | GAGATCTGATTCTGTGAAGGGCGACACGTA | |
| | CTGCTCAGGTTGTGGAGGAAATAATTTGTT | |
| | GGCTGAATATTCCAGCCATTGAAGCTTTGT | |
| | TGCCCATTCATGAGGGAACTCTTCTTTGAT | |
| | CATGTCAAGATACTCCTCCTTAGACGTTGC | |
| | AGTCTGGATAATAGTTCGCCATCGTGCGTC | |
| | AGATTTGCGAGGAGACACCTTATGATCTCG | |
| | GAAATCTCCTCTGGTTTTAATATCTCCGTC | |
| | CTTTGATATGTAATCAAGGACTTGTTTAGA | |
| | GTTTCTAGCTGGCTGGATATTAGGGTGATT | |
| | TCCTTCAAAATCGAAAAAAGAAGGATCCCT | |
| | AATACAAGGTTTTTTATCAAGCTGGATAAG | |
| | AGCATGATAGTGGGTAGTGCCATCTTGATG | |
| | AAGCTCAGAAGCAACACCAAGGAAGAAAAT | |
| | AAGAAAAGGTGTGAGTTTCTCCCAGAGAAA | |
| | CTGGAATAAATCATCTCTTTGAGATGAGCA | |
| | CTTGGGGTAGGTAAGGAAAACATATTTAGA | |
| | TTGGAGTCTGAAGTTCTTGCTAGCAGAAGG | |
| | CATGTTGTTGTGACTCCGAGGGGTTGCCTC | |
| | AAACTCTATCTTATAACCGGCGTGGAGGCA | |
| | TGGAGGCAAGGGCATTTTGGTAATTTAAGT | |
| | AGTTAGTGGAAAATGACGTCATTTACTTAA | |
| | AGACGAAGTCTTGCGACAAGGGGGGCCCAC | |
| | GCCGAATTTTAATATTACCGGCGTGGCCCC | |
| | ACCTTATCGCGAGTGCTTTAGCACGAGCGG | |
| | TCCAGATTTAAAGTAGAAAAGTTCCCGCCC | |
| | ACTAGGGTTAAAGGTGTTCACACTATAAAA | |
| | GCATATACGATGTGATGGTATTTGATGGAG | |
| | CGTATATTGTATCAGGTATTTCCGTCGGAT | |
| | ACGAATTATTCGTACGgccggaccggtccc | |
| | ctaggccggccaattCGAGATCGGCCGCGG | |
| | CTGAGTGGCTCCTTCAATCGTTGCGGTTCT | |
| | GTCAGTTCCAAACGTAAAACGGCTTGTCCC | |
| | GCGTCATCGGCGGGGGTCATAACGTGACTC | |
| | CCTTAATTCTCCGCTCATGATCAGATTGTC | |
| | GTTTCCCGCCTTCAGTTTAAACTATCAGTG | |
| | TTTGACAGGATATATTGGCGGGTAAACCTA | |
| | AGAGAAAAGAGCGTTTATTAGAATAATCGG | |
| | ATATTTAAAAGGGCGTGAAAAGGTTTATCC | |
| | GTTCGTCCATTTGTATGTGCATGCCAACCA | |
| | CAGGGTTCCCCAGATCTGGCGCCGGCCAGC | |
| | GAGACGAGCAAGATTGGCCGCCGCCCGAAA | |
| | CGATCCGACAGCGCGCCCAGCACAGGTGCG | |
| | CAGGCAAATTGCACCAACGCATACAGCGCC | |
| | AGCAGAATGCCATAGTGGGCGGTGACGTCG | |
| | TTCGAGTGAACCAGATCGCGCAGGAGGCCC | |
| | GGCAGCACCGGCATAATCAGGCCGATGCCG | |
| | ACAGCGTCGAGCGCGACAGTGCTCAGAATT | |
| | ACGATCAGGGGTATGTTGGGTTTCACGTCT | |
| | GGCCTCCGGAGACTGTCATACGCGTAAAAA | |
| | GGCCGCGTTGCTGGCGTTTTTCCATAGGCT | |
| | CCGCCCCCCTGACGAGCATCACAAAAATCG | |
| | ACGCTCAAGTCAGAGGTGGCGAAACCCGAC | |
| | AGGACTATAAAGATACCAGGCGTTTCCCCC | |
| | TGGAAGCTCCCTCGTGCGCTCTCCTGTTCC | |
| | GACCCTGCCGCTTACCGGATACCTGTCCGC | |
| | CTTTCTCCCTTCGGGAAGCGTGGCGCTTTC | |
| | TCATAGCTCACGCTGTAGGTATCTCAGTTC | |
| | GGTGTAGGTCGTTCGCTCCAAGCTGGGCTG | |
| | TGTGCACGAACCCCCCGTTCAGCCCGACCG | |
| | CTGCGCCTTATCCGGTAACTATCGTCTTGA | |
| | GTCCAACCCGGTAAGACACGACTTATCGCC | |
| | ACTGGCAGCAGCCACTGGTAACAGGATTAG | |
| | CAGAGCGAGGTATGTAGGCGGTGCTACAGA | |
| | GTTCTTGAAGTGGTGGCCTAACTACGGCTA | |
| | CACTAGAAGGACAGTATTTGGTATCTGCGC | |
| | TCTGCTGAAGCCAGTTACCTTCGGAAAAAG | |
| | AGTTGGTAGCTCTTGATCCGGCAAACAAAC | |
| | CACCGCTGGTAGCGGTGGTTTTTTTGTTTG | |
| | CAAGCAGCAGATTACGCGCAGAAAAAAAGG | |
| | ATCTCAAGAAGATCCTTTGATCTTTTCTAC | |
| | GGGGTCTGACGCTCAGTGGAACGAAAACTC | |
| | ACGTTAAGGGATTTTGGTCATGAGATTATC | |
| | AAAAAGGATCTTCACCTAGATCCTTTTAAA | |
| | TTAAAAATGAAGTTTTAAATCAATCTAAAG | |
| | TATATATGAGTAAACTTGGTCTGCAGTTGC | |
| | CATGTTTTACGGCAGTGAGAGCAGAGATAG | |
| | CGCTGATGTCCGGCGGTGCTTTTGCCGTTA | |
| | CGCACCACCCCGTCAGTAGCTGAACAGGAG | |
| | GGACAGCTGATAGACACAGAAGCCACTGGA | |
| | GCACCTCAAAAACACCATCATACACTAAAT | |
| | CAGTAAGTTGGCAGCATCACCCATAATTGT | |
| | GGTTTCAAAATCGGCTCCGTCGATACTATG | |
| | TTATACGCCAACTTTGAAAACAACTTTGAA | |
| | AAAGCTGTTTTCTGGTATTTAAGGTTTTAG | |
| | AATGCAAGGAACAGTGAATTGGAGTTCGTC | |
| | TTGTTATAATTAGCTTCTTGGGGTATCTTT | |
| | AAATACTGTAGAAAAGAGGAAGGAAATAAT | |
| | AAATGGCTAAAATGAGAATATCACCGGAAT | |
| | TGAAAAAACTGATCGAAAAATACCGCTGCG | |
| | TAAAAGATACGGAAGGAATGTCTCCTGCTA | |
| | AGGTATATAAGCTGGTGGGAGAAAATGAAA | |
| | ACCTATATTTAAAAATGACGGACAGCCGGT | |
| | ATAAAGGGACCACCTATGATGTGGAACGGG | |
| | AAAAGGACATGATGCTATGGCTGGAAGGAA | |
| | AGCTGCCTGTTCCAAAGGTCCTGCACTTTG | |
| | AACGGCATGATGGCTGGAGCAATCTGCTCA | |
| | TGAGTGAGGCCGATGGCGTCCTTTGCTCGG | |
| | AAGAGTATGAAGATGAACAAAGCCCTGAAA | |
| | AGATTATCGAGCTGTATGCGGAGTGCATCA | |
| | GGCTCTTTCACTCCATCGACATATCGGATT | |
| | GTCCCTATACGAATAGCTTAGACAGCCGCT | |
| | TAGCCGAATTGGATTACTTACTGAATAACG | |
| | ATCTGGCCGATGTGGATTGCGAAAACTGGG | |
| | AAGAAGACACTCCATTTAAAGATCCGCGCG | |
| | AGCTGTATGATTTTTTAAAGACGGAAAAGC | |
| | CCGAAGAGGAACTTGTCTTTTCCCACGGCG | |
| | ACCTGGGAGACAGCAACATCTTTGTGAAAG | |
| | ATGGCAAAGTAAGTGGCTTTATTGATCTTG | |
| | GGAGAAGCGGCAGGGCGGACAAGTGGTATG | |
| | ACATTGCCTTCTGCGTCCGGTCGATCAGGG | |
| | AGGATATCGGGGAAGAACAGTATGTCGAGC | |
| | TATTTTTTGACTTACTGGGGATCAAGCCTG | |
| | ATTGGGAGAAAATAAAATATTATATTTTAC | |
| | TGGATGAATTGTTTTAGTACCTAGATGTGG | |
| | CGCAACGATGCCGGCGACAAGCAGGAGCGC | |
| | ACCGACTTCTTCCGCATCAAGTGTTTTGGC | |
| | TCTCAGGCCGAGGCCCACGGCAAGTATTTG | |
| | GGCAAGGGGTCGCTGGTATTCGTGCAGGGC | |
| | AAGATTCGGAATACCAAGTACGAGAAGGAC | |
| | GGCCAGACGGTCTACGGGACCGACTTCATT | |
| | GCCGATAAGGTGGATTATCTGGACACCAAG | |
| | GCACCAGGCGGGTCAAATCAGGAATAAGGG | |
| | CACATTGCCCCGGCGTGAGTCGGGGCAATC | |
| | CCGCAAGGAGGGTGAATGAATCGGACGTTT | |
| | GACCGGAAGGCATACAGGCAAGAACTGATC | |
| | GACGCGGGGTTTTCCGCCGAGGATGCCGAA | |
| | ACCATCGCAAGCCGCACCGTCATGCGTGCG | |
| | CCCCGCGAAACCTTCCAGTCCGTCGGCTCG | |
| | ATGGTCCAGCAAGCTACGGCCAAGATCGAG | |
| | CGCGACAGCGTGCAACTGGCTCCCCCTGCC | |
| | CTGCCCGCGCCATCGGCCGCCGTGGAGCGT | |
| | TCGCGTCGTCTCGAACAGGAGGCGGCAGGT | |
| | TTGGCGAAGTCGATGACCATCGACACGCGA | |
| | GGAACTATGACGACCAAGAAGCGAAAAACC | |
| | GCCGGCGAGGACCTGGCAAAACAGGTCAGC | |
| | GAGGCCAAGCAGGCCGCGTTGCTGAAACAC | |
| | ACGAAGCAGCAGATCAAGGAAATGCAGCTT | |
| | TCCTTGTTCGATATTGCGCCGTGGCCGGAC | |
| | ACGATGCGAGCGATGCCAAACGACACGGCC | |
| | CGCTCTGCCCTGTTCACCACGCGCAACAAG | |
| | AAAATCCCGCGCGAGGCGCTGCAAAACAAG | |
| | GTCATTTTCCACGTCAACAAGGACGTGAAG | |
| | ATCACCTACACCGGCGTCGAGCTGCGGGCC | |
| | GACGATGACGAACTGGTGTGGCAGCAGGTG | |
| | TTGGAGTACGCGAAGCGCACCCCTATCGGC | |
| | GAGCCGATCACCTTCACGTTCTACGAGCTT | |
| | TGCCAGGACCTGGGCTGGTCGATCAATGGC | |
| | CGGTATTACACGAAGGCCGAGGAATGCCTG | |
| | TCGCGCCTACAGGCGACGGCGATGGGCTTC | |
| | ACGTCCGACCGCGTTGGGCACCTGGAATCG | |
| | GTGTCGCTGCTGCACCGCTTCCGCGTCCTG | |
| | GACCGTGGCAAGAAAACGTCCCGTTGCCAG | |
| | GTCCTGATCGACGAGGAAATCGTCGTGCTG | |
| | TTTGCTGGCGACCACTACACGAAATTCATA | |
| | TGGGAGAAGTACCGCAAGCTGTCGCCGACG | |
| | GCCCGACGGATGTTCGACTATTTCAGCTCG | |
| | CACCGGGAGCCGTACCCGCTCAAGCTGGAA | |
| | ACCTTCCGCCTCATGTGCGGATCGGATTCC | |
| | ACCCGCGTGAAGAAGTGGCGCGAGCAGGTC | |
| | GGCGAAGCCTGCGAAGAGTTGCGAGGCAGC | |
| | GGCCTGGTGGAACACGCCTGGGTCAATGAT | |
| | GACCTGGTGCATTGCAAACGCTAGGGCCTT | |
| | GTGGGGTCAGTTCCGGCTGGGGGTTCAGCA | |
| | GCCAGCGCTTTACTGGCATTTCAGGAACAA | |
| | GCGGGCACTGCTCGACGCACTTGCTTCGCT | |
| | CAGTATCGCTCGGGACGCACGGCGCGCTCT | |
| | ACGAACTGCCGATAAACAGAGGATTAAAAT | |
| | TGACAATTCAATGGCAAGGACTGCCAGCGC | |
| | TGCCATTTTTGGGGTGAGGCCGTTCGCGGC | |
| | CGAGGGGCGCAGCCCCTGGGGGGATGGGAG | |
| | GCCCGCGTTAGCGGGCCGGGAGGGTTCGAG | |
| | AAGGGGGGGCACCCCCCTTCGGCGTGCGCG | |
| | GTCACGCGCACAGGGCGCAGCCCTGGTTAA | |
| | AAACAAGGTTTATAAATATTGGTTTAAAAG | |
| | CAGGTTAAAAGACAGGTTAGCGGTGGCCGA | |
| | AAAACGGGCGGAAACCCTTGCAAATGCTGG | |
| | ATTTTCTGCCTGTGGACAGCCCCTCAAATG | |
| | TCAATAGGTGCGCCCCTCATCTGTCAGCAC | |
| | TCTGCCCCTCAAGTGTCAAGGATCGCGCCC | |
| | CTCATCTGTCAGTAGTCGCGCCCCTCAAGT | |
| | GTCAATACCGCAGGGCACTTATCCCCAGGC | |
| | TTGTCCACATCATCTGTGGGAAACTCGCGT | |
| | AAAATCAGGCGTTTTCGCCGATTTGCGAGG | |
| | CTGGCCAGCTCCACGTCGCCGGCCGAAATC | |
| | GAGCCTGCCCCTCATCTGTCAACGCCGCGC | |
| | CGGGTGAGTCGGCCCCTCAAGTGTCAACGT | |
| | CCGCCCCTCATCTGTCAGTGAGGGCCAAGT | |
| | TTTCCGCGAGGTATCCACAACGCCGGCGGC | |
| | CGCGGTGTCTCGCACACGGCTTCGACGGCG | |
| | TTTCTGGCGCGTTTGCAGGGCCATAGACGG | |
| | CCGCCAGCCCAGCGGCGAGGGCAACCAGCC | |
| | CGGTGAGCGTCGCAAAGGCGCTCGGTCTTG | |
| | CCTTGCTCGTCGAGATCTGGGGTCGATCAG | |
| | CCGGGGATGCATCAGGCCGACAGTCGGAAC | |
| | TTCGGGTCCCCGACCTGTACCATTCGGTGA | |
| | GCAATGGATAGGGGAGTTGATATCGTCAAC | |
| | GTTCACTTCTAAAGAAATAGCGCCACTCAG | |
| | CTTCCTCAGCGGCTTTATCCAGCGATTTCC | |
| | TATTATGTCGGCATAGTTCTCAAGATCGAC | |
| | AGCCTGTCACGGTTAAGCGAGAAATGAATA | |
| | AGAAGGCTGATAATTCGGATCTCTGCGAGG | |
| | GAGATGATATTTGATCACAGGCAGCAACGC | |
| | TCTGTCATCGTTACAATCAACATGCTACCC | |
| | TCCGCGAGATCATCCGTGTTTCAAACCCGG | |
| | CAGCTTAGTTGCCGTTCTTCCGAATAGCAT | |
| | CGGTAACATGAGCAAAGTCTGCCGCCTTAC | |
| | AACGGCTCTCCCGCTGACGCCGTCCCGGAC | |
| | TGATGGGCTGCCTGTATCGAGTGGTGATTT | |
| | TGTGCCGAGCTGCCGGTCGGGGAGCTGTTG | |
| | GCTGGCTGGTGGCAGGATATATTGTGGTGT | |
| | AAACAAATTGACGCTTAGACAACTTAATAA | |
| | CACATTGCGGACGTTTTTAATGTACTGGGG | |
| | TGGTTTTTCTTTTCACCAGTGAGACGGGCA | |
| | ACAGCTGATTGCCCTTCACCGCCTGGCCCT | |
| | GAGAGAGTTGCAGCAAGCGGTCCACGCTGG | |
| | TTTGCCCCAGCAGGCGAAAATCCTGTTTGA | |
| | TGGTGGTTCCGAAATCGGCAAAATCCCTTA | |
| | TAAATCAAAAGAATAGCCCGAGATAGGGTT | |
| | GAGTGTTGTTCCAGTTTGGAACAAGAGTCC | |
| | ACTATTAAAGAACGTGGACTCCAACGTCAA | |
| | AGGGCGAAAAACCGTCTATCAGGGCGATGG | |
| | CCCACTACGTGAACCATCACCCAAATCAAG | |
| | TTTTTTGGGGTCGAGGTGCCGTAAAGCACT | |
| | AAATCGGAACCCTAAAGGGAGCCCCCGATT | |
| | TAGAGCTTGACGGGGAAAGCCGGCGAACGT | |
| | GGCGAGAAAGGAAGGGAAGAAAGCGAAAGG | |
| | AGCGGGCGCCATTCAGGCTGCGCAACTGTT | |
| | GGGAAGGG | |
| 2 | TGGGATGGTCTTGTATCATCCTTTTCTTGG | CHIKV152H_{C} |
| | TTGCAACAGCTACTGGTGTTCATTCTCAAG | |
| | TTCAATTGCAGCAGCCTGGTGCAGCTCTCG | |
| | TGAAGCCAGGTGCTTCAGCAATGATGTCTT | |
| | GTAAGGCTTCTGGTTACACATTCACAAGCT | |
| | ATTGGATTACATGGGTGAAACAAAGACCTG | |
| | GTCAGGGTCTTGAGTGGATAGGAGATATTT | |
| | ATCCCGGAACTGGAAGAACTATCTACAAAG | |
| | AGAAGTTTAAAACCAAGGCTACTCTGACTG | |
| | TAGATACCAGTTCTTCTACTGCTTTTATGC | |
| | AACTTAATTCTTTGACATCAGAAGATAGTG | |
| | CTGTGTACTATTGTGCTAGGGGGTATGGCT | |
| | CACCATATTACGCTTTAGACTATTGGGGAC | |
| | AAGGTACTTCCGTTACTGTGTCTTCTGCTA | |
| | GCACCAAGGGACCTTCTGTTTTTCCACTTG | |
| | CTCCTTCTTCTAAGTCTACTTCTGGTGGAA | |
| | CTGCTGCTTTGGGTTGTTTGGTGAAAGATT | |
| | ACTTTCCTGAGCCAGTGACCGTTTCTTGGA | |
| | ACTCAGGTGCTCTTACATCTGGTGTTCATA | |
| | CTTTCCCAGCTGTTCTTCAATCTTCAGGAC | |
| | TTTACTCACTTTCTTCTGTTGTTACCGTTC | |
| | CTTCTTCAAGCTTGGGCACTCAGACCTACA | |
| | TCTGCAATGTGAATCACAAACCCAGCAACA | |
| | CCAAGGTTGACAAGAAAGTTGAGCCCAAGT | |
| | CTTGTGACAAGACTCATACGTGTCCACCGT | |
| | GCCCAGCACCTGAACTTCTTGGAGGACCGT | |
| | CAGTCTTCTTGTTTCCTCCAAAGCCTAAGG | |
| | ATACCTTGATGATCTCCAGGACTCCTGAAG | |
| | TCACATGTGTAGTTGTGGATGTGAGCCATG | |
| | AAGATCCTGAGGTGAAGTTCAACTGGTATG | |
| | TGGATGGTGTGGAAGTGCACAATGCCAAGA | |
| | CAAAGCCGAGA | |
| | GAGGAACAGTACAACAGCACGTACAGGGTT | |
| | GTCTCAGTTCTCACTGTTCTCCATCAAGAT | |
| | TGGTTGAATGGCAAAGAGTACAAGTGCAAG | |
| | GTGTCCAACAAAGCCCTCCCAGCCCCCATT | |
| | GAGAAGACCATTTCCAAAGCGAAAGGGCAA | |
| | CCCCGTGAACCACAAGTGTACACACTTCCT | |
| | CCATCTCGCGATGAACTGACCAAGAACCAG | |
| | GTCAGCTTGACTTGCCTGGTGAAAGGCTTC | |
| | TATCCCTCTGACATAGCTGTAGAGTGGGAG | |
| | AGCAATGGGCAACCGGAGAACAACTACAAG | |
| | ACTACACCTCCCGTTCTCGATTCTGACGGC | |
| | TCCTTCTTCCTCTACAGCAAGCTCACAGTG | |
| | GACAAGAGCAGGTGGCAACAAGGGAATGTC | |
| | TTCTCATGCTCCGTGATGCATGAGGCTCTT | |
| | CACAATCACTACACACAGAAGAGTCTCTCC | |
| | TTGTCTCCGGGTAAATGA | |
| 3 | MGWSCIILFLVATATGVHSQVQLQQPGAAL | CHIKV152H_{C} |
| | VKPGASAMMSCKASGYTFTSYWITWVKQRP | |
| | GQGLEWIGDIYPGTGRTIYKEKFKTKATLT | |
| | VDTSSSTAFMQLNSLTSEDSAVYYCARGYG | |
| | SPYYALDYWGQGTSVTVSSASTKGPSVFPL | |
| | APSSKSTSGGTAALGCLVKDYFPEPVTVSW | |
| | NSGALTSGVHTFPAVLQSSGLYSLSSVVTV | |
| | PSSSLGTQTYICNVNHKPSNTKVDKKVEPK | |
| | SCDKTHTCPPCPAPELLGGPSVFLFPPKPK | |
| | DTLMISRTPEVTCVVVDVSHEDPEVKFNWY | |
| | VDGVEVHNAKTKPREEQYNSTYRVVSVLTV | |
| | LHQDWLNGKEYKCKVSNKALPAPIEKTISK | |
| | AKGQPREPQVYTLPPSRDELTKNQVSLTCL | |
| | VKGFYPSDIAVEWESNGQPENNYKTTPPVL | |
| | DSDGSFFLYSKLTVDKSRWQQGNVFSCSVM | |
| | HEALHNHYTQKSLSLSPGK | |
| 4 | ATGGGATGGTCTTGTATCATCCTTTTCTTG | CHIKV152L_{C} |
| | GTTGCAACAGCTACTGGTGTTCATTCTGAT | |
| | ATCGTTTTAACACAATCTCCAGCATCTTTG | |
| | GCTGTTTCTCAAGGACAAAGGGCTACTATC | |
| | AGTTGCAAGGCTTCTCAATCAGTGGACTAT | |
| | GACGGTGATTCTTATGTTAACTGGTACCAA | |
| | CAGAAACCTGGACAGTCCCCAAAACTTCTT | |
| | ATCTACGACGCATCTAATCTCGAATCTGGA | |
| | ATACCAGCCAGATTTTCTGGATCTGGATCT | |
| | GGAACAGATTTCACTCTTAACATCCATCCT | |
| | GTC | |
| | GAGGAAGAGGATGTTGCAACTTATTACTGT | |
| | CAGGAATCAAATGAAGATCCACGTACTTTC | |
| | GGTGGAGGAACTAAGTTGGAGATCAAAGCT | |
| | AGCAGAACTGTTGCTGCACCATCTGTTTTC | |
| | ATCTTCCCTCCATCTGATGAGCAGTTGAAA | |
| | TCTGGAACTGCTTCTGTTGTGTGCCTTCTT | |
| | AATAACTTCTATCCTAGAGAGGCTAAAGTT | |
| | CAGTGGAAGGTGGATAACGCACTTCAATCT | |
| | GGTAACTCTCAAGAGTCTGTTACAGAGCAA | |
| | GATTCTAAGGACTCAACTTACTCTCTTTCA | |
| | TCTACACTTACTTTGTCAAAAGCAGATTAC | |
| | GAGAAACACAAAGTTTACGCATGCGAAGTT | |
| | ACTCATCAAGGACTTTCTTCACCAGTTACA | |
| | AAGTCTTTCAATAGAGGAGAGTGTTAA | |
| 5 | MGWSCIILFLVATATGVHSDIVLTQSPASL | CHIKV152L_{C} |
| | AVSQGQRATISCKASQSVDYDGDSYVNWYQ | |
| | QKPGQSPKLLIYDASNLESGIPARFSGSGS | |
| | GTDFTLNIHPVEEEDVATYYCQESNEDPRT | |
| | FGGGTKLEIKASRTVAAPSVFIFPPSDEQL | |
| | KSGTASVVCLLNNFYPREAKVQWKVDNALQ | |
| | SGNSQESVTEQDSKDSTYSLSSTLTLSKAD | |
| | YEKHKVYACEVTHQGLSSPVTKSFNRGEC* | |
| 6 | ATGGGATGGTCTTGTATCATCCTTTTCTTG | CHIKV166H_{C} |
| | GTTGCAACAGCTACTGGTGTTCATTCTGAA | |
| | GTTAGGTTGGTGGAATCAGGAGGCGGTCTT | |
| | GAGCAGCCAGGGGGTTCACTGAAGCTATCT | |
| | TGTGCAGCTTCTGGTTTTACATTCAGTGAT | |
| | TACTTTATGTATTGGGTGAGACAAACCCCT | |
| | GAGAAACGTCTTGAGTGGGTCGCCTACATT | |
| | AGTAATGGGGGAATTTCCACTTTCTATAGC | |
| | GATGCAGTAAAAGGAAGATTCACTATATCC | |
| | AGAGACAACGCTAGAAATACTCTTTATCTC | |
| | CAAATGTCTAGATTGAAGTCAGAAGATACT | |
| | GCTATCTACTATTGTGTTAGGCAGGTATAT | |
| | GGCCAGGGATACTTTGACTATTGGGGACAA | |
| | GGTACTACTTTAGCAGTTTCTTCTGCTAGC | |
| | ACCAAGGGACCTTCTGTTTTTCCACTTGCT | |
| | CCTTCTTCTAAGTCTACTTCTGGTGGAACT | |
| | GCTGCTTTGGGTTGTTTGGTGAAAGATTAC | |
| | TTTCCTGAGCCAGTGACCGTTTCTTGGAAC | |
| | TCAGGTGCTCTTACATCTGGTGTTCATACT | |
| | TTCCCAGCTGTTCTTCAATCTTCAGGACTT | |
| | TACTCACTTTCTTCTGTTGTTACCGTTCCT | |
| | TCTTCAAGCTTGGGCACTCAGACCTACATC | |
| | TGCAATGTGAATCACAAACCCAGCAACACC | |
| | AAGGTTGACAAGAAAGTTGAGCCCAAGTCT | |
| | TGTGACAAGACTCATACGTGTCCACCGTGC | |
| | CCAGCACCTGAACTTCTTGGAGGACCGTCA | |
| | GTCTTCTTGTTTCCTCCAAAGCCTAAGGAT | |
| | ACCTTGATGATCTCCAGGACTCCTGAAGTC | |
| | ACATGTGTAGTTGTGGATGTGAGCCATGAA | |
| | GATCCTGAGGTGAAGTTCAACTGGTATGTG | |
| | GATGGTGTGGAAGTGCACAATGCCAAGACA | |
| | AAGCCGAGAGAGGAACAGTACAACAGCACG | |
| | TACAGGGTTGTCTCAGTTCTCACTGTTCTC | |
| | CATCAAGATTGGTTGAATGGCAAAGAGTAC | |
| | AAGTGCAAGGTGTCCAACAAAGCCCTCCCA | |
| | GCCCCCATTGAGAAGACCATTTCCAAAGCG | |
| | AAAGGGCAACCCCGTGAACCACAAGTGTAC | |
| | ACACTTCCTCCATCTCGCGATGAACTGACC | |
| | AAGAACCAGGTCAGCTTGACTTGCCTGGTG | |
| | AAAGGCTTCTATCCCTCTGACATAGCTGTA | |
| | GAGTGGGAGAGCAATGGGCAACCGGAGAAC | |
| | AACTACAAGACTACACCTCCCGTTCTCGAT | |
| | TCTGACGGCTCCTTCTTCCTCTACAGCAAG | |
| | CTCACAGTGGACAAGAGCAGGTGGCAACAA | |
| | GGGAATGTCTTCTCATGCTCCGTGATGCAT | |
| | GAGGCTCTTCACAATCACTACACACAGAAG | |
| | AGTCTCTCCTTGTCTCCGGGTAAATGA | |
| 7 | MGWSCIILFLVATATGVHSEVRLVESGGGL | CHIKV166H_{C} |
| | EQPGGSLKLSCAASGFTFSDYFMYWVRQTP | |
| | EKRLEWVAYISNGGISTFYSDAVKGRFTIS | |
| | RDNARNTLYLQMSRLKSEDTAIYYCVRQVY | |
| | GQGYFDYWGQGTTLAVSSASTKGPSVFPLA | |
| | PSSKSTSGGTAALGCLVKDYFPEPVTVSWN | |
| | SGALTSGVHTFPAVLQSSGLYSLSSVVTVP | |
| | SSSLGTQTYICNVNHKPSNTKVDKKVEPKS | |
| | CDKTHTCPPCPAPELLGGPSVFLFPPKPKD | |
| | TLM1SRTPEVTCVVVDVSHEDPEVKFNWYV | |
| | DGVEVHNAKTKPREEQYNSTYRVVSVLTVL | |
| | HQDWLNGKEYKCKVSNKALPAPIEKTISKA | |
| | KGQPREPQVYTLPPSRDELTKNQVSLTCLV | |
| | KGFYPSDIAVEWESNGQPENNYKTTPPVLD | |
| | SDGSFFLYSKLTVDKSRWQQGNVFSCSVMH | |
| | EALHNHYTQKSLSLSPGK* | |
| 8 | ATGGGATGGTCTTGTATCATCCTTTTCTTG | CHKV166L_{C} |
| | GTTGCAACAGCTACTGGTGTTCATTCTCAA | |
| | ATCGTTCTGATTCAATCTCCAGCAATTATG | |
| | TCTGCTTCTCTTGGAGAGAGGGTGACTATG | |
| | ACTTGCACCGCTAGTAGCTCTGTTTCAAGT | |
| | TCTTATTTGCACTGGTACCAACAGAAACCT | |
| | GGATCATCACCAAAACTTTGGATCTACTCC | |
| | TCATTTTCTTTAGCATCTGGAGTTCCAGCC | |
| | AGATTTTCTGGATCTGGATCTGGAACATCC | |
| | TATAGCCTTACTATCTCAACAATGGAAGCT | |
| | GAGGATGCTGCAACATATTACTGTCATCAG | |
| | TATCTCCGTTCCCCATGGACTTTCGGTGGA | |
| | GGAAGTAAGTTGGAGATCAAAGCTAGCAGA | |
| | ACTGTTGCTGCACCATCTGTTTTCATCTTC | |
| | CCTCCATCTGATGAGCAGTTGAAATCTGGA | |
| | ACTGCTTCTGTTGTGTGCCTTCTTAATAAC | |
| | TTCTATCCTAGAGAGGCTAAAGTTCAGTGG | |
| | AAGGTGGATAACGCACTTCAATCTGGTAAC | |
| | TCTCAAGAGTCTGTTACAGAGCAAGATTCT | |
| | AAGGACTCAACTTACTCTCTTTCATCTACA | |
| | CTTACTTTGTCAAAAGCAGATTACGAGAAA | |
| | CACAAAGTTTACGCATGCGAAGTTACTCAT | |
| | CAAGGACTTTCTTCACCAGTTACAAAGTCT | |
| | TTCAATAGAGGAGAGTGTTAATGA | |
| 9 | MGWSCIILFLVATATGVHSQIVLIQSPAIM | CHKV166L_{C} |
| | SAS0LGERVTMTCTASSSVSSSYLHWYQQK | |
| | PGSSPKLWIYSSFSLASGVPARFSGSGSGT | |
| | SYSLTISTMEAEDAATYYCHQYLRSPWTFG | |
| | GGSKLEIKASRTVAAPSVFIFPPSDEQLKS | |
| | GTASVVCLLNNFYPREAKVQWKVDNALQSG | |
| | NSQESVTEQDSKDSTYSLSSTLTLSKADYE | |
| | KHKVYACEVTHQGLSSPVTKSFNRGEC* | |
| 10 | GAATTCACAATGGGATGGTCTTGTATCATC | 152V_{H} |
| | CTTTTCTTGGTTGCAACAGCTACTGGTGTT | |
| | CATTCTCAAGTTCAATTGCAGCAGCCTGGT | |
| | GCAGCTCTCGTGAAGCCAGGTGCTTCAGCA | |
| | ATGATGTCTTGTAAGGCTTCTGGTTACACA | |
| | TTCACAAGCTATTGGATTACATGGGTGAAA | |
| | CAAAGACCTGGTCAGGGTCTTGAGTGGATA | |
| | GGAGATATTTATCCCGGAACTGGAAGAACT | |
| | ATCTACAAAGAGAAGTTTAAAACCAAGGCT | |
| | ACTCTGACTGTAGATACCAGTTCTTCTACT | |
| | GCTTTTATGCAACTTAATTCTTTGACATCA | |
| | GAAGATAGTGCTGTGTACTATTGTGCTAGG | |
| | GGGTATGGCTCACCATATTACGCTTTAGAC | |
| | TATTGGGGACAAGGTACTTCCGTTACTGTG | |
| | TCTTCTGCTAGC | |
| 11 | MGWSCIILFLVATATGVHSQVQLQQPGAAL | 152V_{H} |
| | VKPGASAMMSCKASGYTFTSYWITWVKQRP | |
| | GQGLEWIGDIYPGTGRTIYKEKFKTKATLT | |
| | VDTSSSTAFMQLNSLTSEDSAVYYCARGYG | |
| | SPYYALDYWGQGTSVTVSSAS | |
| 12 | GAATTCACAATGGGATGGTCTTGTATCATC | 152V_{L} |
| | CTTTTCTTGGTTGCAACAGCTACTGGTGTT | |
| | CATTCTGATATCGTTTTAACACAATCTCCA | |
| | GCATCTTTGGCTGTTTCTCAAGGACAAAGG | |
| | GCTACTATCAGTTGCAAGGCTTCTCAATCA | |
| | GTGGACTATGACGGTGATTCTTATGTTAAC | |
| | TGGTACCAAC | |
| | AGAAACCTGGACAGTCCCCAAAACTTCTTA | |
| | TCTACGACGCATCTAATCTCGAATCTGGAA | |
| | TACCAGCCAGATTTTCTGGATCTGGATCTG | |
| | GAACAGATTTCACTCTTAACATCCATCCTG | |
| | TCGAGGAAGAGGATGTTGCAACTTATTACT | |
| | GTCAGGAATCAAATGAAGATCCACGTACTT | |
| | TCGGTGGAGGAACTAAGTTGGAGATCAAA | |
| 13 | MGWSCIILFLVATATGVHSDIVLTQSPASL | 152V_{L} |
| | AVSQGQRATISCKASQSVDYDGDSYVNWYQ | |
| | QKPGQSPKLLIYDASNLESGIPARFSGSGS | |
| | GTDFTLNIHPVEEEDVATYYCQESNEDPRT | |
| | FGGGTKLEIK | |
| 14 | GAATTCACAATGGGATGGTCTTGTATCATC | 166V_{H} |
| | CTTTTCTTGGTTGCAACAGCTACTGGTGTT | |
| | CATTCTGAAGTTAGGTTGGTGGAATCAGGA | |
| | GGCGGTCTTGAGCAGCCAGGGGGTTCACTG | |
| | AAGCTATCTTGTGCAGCTTCTGGTTTTACA | |
| | TTCAGTGATTACTTTATGTATTGGGTGAGA | |
| | CAAACCCCTGAGAAACGTCTTGAGTGGGTC | |
| | GCCTACATTAGTAATGGGGGAATTTCCACT | |
| | TTCTATAGCGATGCAGTAAAAGGAAGATTC | |
| | ACTATATCCAGAGACAACGCTAGAAATACT | |
| | CTTTATCTCCAAATGTCTAGATTGAAGTCA | |
| | GAAGATACTGCTATCTACTATTGTGTTAGG | |
| | CAGGTATATGGCCAGGGATACTTTGACTAT | |
| | TGGGGACAAGGTACTACTTTAGCAGTTTCT | |
| | TCTGCTAGC | |
| 15 | MGWSCIILFLVATATGVHSEVRLVESGGGL | 166V_{H} |
| | EQPGGSLKLSCAASGFTFSDYFMYWVRQTP | |
| | EKRLEWVAYISNGGISTFYSDAVKGRFTIS | |
| | RDNARNTLYLQMSRLKSEDTAIYYCVRQVY | |
| | GQGYFDYWGQGTTLAVSSAS | |
| 16 | GAATTCACAATGGGATGGTCTTGTATCATC | 166V_{L} |
| | CTTTTCTTGGTTGCAACAGCTACTGGTGTT | |
| | CATTCTCAAATCGTTCTGATTCAATCTCCA | |
| | GCAATTATGTCTGCTTCTCTTGGA | |

What is claimed is:

1. A kit comprising:
a glycosylated IgG monoclonal antibody having specificity to Chikungunya virus (CHIKV) E1 or E2 protein, comprising a heavy chain and a light chain pair selected from:
(i) a heavy chain comprising the amino acid sequence of SEQ. ID NO: 3; and a light chain comprising the amino acid sequence of SEQ. ID NO: 5;
(ii) a heavy chain comprising the amino acid sequence of SEQ. ID NO: 7; and a light chain comprising the amino acid sequence of SEQ. ID NO: 9;
(iii) a heavy chain variable domain comprising the amino acid sequence of SEQ ID NO: 11; and a light chain variable domain comprising the amino acid sequence of SEQ ID NO: 13; or
(iv) a heavy chain variable domain comprising the amino acid sequence of SEQ ID NO: 15; and a light chain variable domain comprising the amino acid sequence of SEQ ID NO: 17;
wherein the Fc domain of the heavy chain is characterized by an N-linked glycan profile comprising an N-linked GlcNAc2Man3GlcNAc2 (GnGn) core glycan (Man=Mannose, GlcNAc=N-acetylglucosamine) with more than about 95% uniformity.

2. The kit of claim 1, wherein the antibody is produced in a plant, a plant fragment, or a plant cell.

3. The kit of claim 1, wherein the plant is ΔXFT N. benthamiana.

4. The kit of claim 1, wherein the antibody is a human antibody that does not contribute to antibody-dependent enhancement of virus infection.

5. A kit comprising:
(a) a composition comprising a prophylactically or therapeutically effective amount of a glycosylated monoclonal antibody having specificity to CHIKV E1 or E2 protein, wherein the antibody comprises a heavy chain and a light chain pair selected from:
(i) a heavy chain comprising the amino acid sequence of SEQ. ID NO: 3; and a light chain comprising the amino acid sequence of SEQ. ID NO: 5;
(ii) a heavy chain comprising the amino acid sequence of SEQ. ID NO: 7; and a light chain comprising the amino acid sequence of SEQ. ID NO: 9;
(iii) a heavy chain variable domain comprising the amino acid sequence of SEQ ID NO: 11; and a light chain variable domain comprising the amino acid sequence of SEQ ID NO: 13; or
(iv) a heavy chain variable domain comprising the amino acid sequence of SEQ ID NO: 15; and a light chain variable domain comprising the amino acid sequence of SEQ ID NO: 17; and
(b) instructions for administering (a) for the prevention or treatment of Chikungunya;
wherein the Fc domain of the heavy chain is characterized by an N-linked glycan profile comprising an N-linked GlcNAc2Man3GlcNAc2 (GnGn) core glycan (Man=Mannose, GlcNAc=N-acetylglucosamine) with more than about 95% uniformity.

6. The kit of claim 5, wherein the composition comprises a plant, plant fragment, plant cell or plant extract comprising the monoclonal antibody having specificity to CHIKV E1 or E2 protein.

7. The kit of claim 6, wherein the plant is ΔXFT N. benthamiana.

8. The kit of claim 5, wherein the composition further comprises excipients selected from the group consisting of compatible dispersing agents, buffers, surfactants, preservatives, solubilizing agents, isotonicity agents, and stabilizing agents.

9. A method of treating and preventing an infection by CHIKV in a subject in need thereof, comprising: administering a glycosylated IgG monoclonal antibody having specificity to Chikungunya virus (CHIKV) E1 or E2 protein, comprising a heavy chain and a light chain pair selected from:
(i) a heavy chain comprising the amino acid sequence of SEQ. ID NO: 3; and a light chain comprising the amino acid sequence of SEQ. ID NO: 5;
(ii) a heavy chain comprising the amino acid sequence of SEQ. ID NO: 7; and a light chain comprising the amino acid sequence of SEQ. ID NO: 9;
(iii) a heavy chain variable domain comprising the amino acid sequence of SEQ ID NO: 11; and a light chain variable domain comprising the amino acid sequence of SEQ ID NO: 13; or
(iv) a heavy chain variable domain comprising the amino acid sequence of SEQ ID NO: 15; and a light chain variable domain comprising the amino acid sequence of SEQ ID NO: 17;
wherein the Fc domain of the heavy chain is characterized by an N-linked glycan profile comprising an N-linked GlcNAc2Man3GlcNAc2 (GnGn) core glycan (Man=Mannose, GlcNAc=N-acetylglucosamine) with more than about 95% uniformity.

10. The method of claim 9, wherein the antibody does not contribute to antibody-dependent enhancement of virus infection.

11. The method of claim 9, wherein the antibody is produced in a plant, a plant fragment, or a plant cell.

12. The method of claim 11, wherein the plant is ΔXFT N. benthamiana.


SEQUENCE LISTING (17 sequences)

SEQ ID NO: 1 - DNA, 15128 bp, synthetic construct
  misc_feature 1..15128: note = SYNTHESIZED
  source 1..15128: mol_type = other DNA
cgatcggtcgattcatagaagattagatttttcatagtatttttttaaagtaaacctttaactacggttaggacacttttaagttaaatttaatttgaacccttaaattaatttttaaaatagataaatatcaatcatcctgatatgcttttgaaaaaatgaatgagaaagatgattcaattaaggccacattttaatcatgactaaaataatatacagtataatttcatatatatttgctttaaaaaaaaattgacaatccattcgtttctagcaataaatttcttcaaccacaaatatattaaagataactacggcatagaaacaaaaatctatgaagaatttttgtatacttcatatgaaattaaaaaaaacttcattgaacatcaaaataataataataatcataaactcctcaatatttatattcctagcttcttgaattaaattgtttacatattcaacgatgtaaaaaattatttctctatctattttccttatatcatgcatggtttcacatatatcaaaggataaaagcaatctatgtaaattatctcactttattaagttttctatctgaattattgagaacgtagatttctttttgcactatcccccaataattagcaaaacacacctagactagatttgttttgctaacccaattgatattaattatatatgattaatatttatatgtatatggaattggttaataaaatgcatctggttcatcaaagaattataaagacacgtgacattcatttaggataagaaatatggatgatctctttctcttattcagataattagtaattacacataacacacaactttgatgcccacattatagtgattagcatgtcactatgtgtgcatccttttatttcatacattaattaacttggccaatccagaagatggacaagtctagggtcacattgcagggtactctagcttactcgccttctttttcgaaggtttgagtaccttcagggcatcctcttgatacattactttccacttcgattggggcaagctgtagcagttcttgcttagaccgaattgccatctcacagagatgctgaagagttcgcgaccctccagaaacggtgatactaactcctcgaaaccgaatactataggtacatccgatctggtcgaaaccgaaaaatcgagatgctgcatagttaaccgaatctcccgtccaagatccaaggactctgtgcagtgaagcttccgtcctgtcgtatctgagatatctcttaaatacaactttcccgaaaccccagctttccttgaaaccaaggggattatcttgattcgaattcgtctcatcgttatgtagccgccactcagtccaactcggactttcgtcaggaagtttgaagggagaagttgtacctcctgatcctccatcccaacgttcactgttagcttgttccctagcgtcgtttccttgtatagctcgttccatggattgtaaatagtaattgtaatgttgtttgttgtttgttgttgttggtaattgttgtaaaaatacgctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctggcgcgccgctctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacgaccctcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaaacaaacaaaatcaacaaatatagaaaataacgcatttccaattctttgaaatttctgcaacatctagcgagaccaacaacggtctctagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggtaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattccaaagaattatagaagggatcggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaaacaaacaaaatcaacaaatatagaaaataacgcatttccaattctttgaaatttctgcaacatctagaggatccccgggtaccgagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggtaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaactcttctttgatcatgtcaagatactcctccttagacgttgcagtctggataatagttcgccatcgtgcgtcagatttgcgaggagacaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggaccggtcccctaggccggccaattcgagatcggccgcggctgagtggctccttcaatcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggagactgtcatacgcgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcgagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaaggg

SEQ ID NO: 2 - DNA, 1409 bp, synthetic construct
  misc_feature 1..1409: note = SYNTHESIZED
  source 1..1409: mol_type = other DNA
tgggatggtcttgtatcatccttttcttggttgcaacagctactggtgttcattctcaagttcaattgcagcagcctggtgcagctctcgtgaagccaggtgcttcagcaatgatgtcttgtaaggcttctggttacacattcacaagctattggattacatgggtgaaacaaagacctggtcagggtcttgagtggataggagatatttatcccggaactggaagaactatctacaaagagaagtttaaaaccaaggctactctgactgtagataccagttcttctactgcttttatgcaacttaattctttgacatcagaagatagtgctgtgtactattgtgctagggggtatggctcaccatattacgctttagactattggggacaaggtacttccgttactgtgtcttctgctagcaccaagggaccttctgtttttccacttgctccttcttctaagtctacttctggtggaactgctgctttgggttgtttggtgaaagattactttcctgagccagtgaccgtttcttggaactcaggtgctcttacatctggtgttcatactttcccagctgttcttcaatcttcaggactttactcactttcttctgttgttaccgttccttcttcaagcttgggcactcagacctacatctgcaatgtgaatcacaaacccagcaacaccaaggttgacaagaaagttgagcccaagtcttgtgacaagactcatacgtgtccaccgtgcccagcacctgaacttcttggaggaccgtcagtcttcttgtttcctccaaagcctaaggataccttgatgatctccaggactcctgaagtcacatgtgtagttgtggatgtgagccatgaagatcctgaggtgaagttcaactggtatgtggatggtgtggaagtgcacaatgccaagacaaagccgagagaggaacagtacaacagcacgtacagggttgtctcagttctcactgttctccatcaagattggttgaatggcaaagagtacaagtgcaaggtgtccaacaaagccctcccagcccccattgagaagaccatttccaaagcgaaagggcaaccccgtgaaccacaagtgtacacacttcctccatctcgcgatgaactgaccaagaaccaggtcagcttgacttgcctggtgaaaggcttctatccctctgacatagctgtagagtgggagagcaatgggcaaccggagaacaactacaagactacacctcccgttctcgattctgacggctccttcttcctctacagcaagctcacagtggacaagagcaggtggcaacaagggaatgtcttctcatgctccgtgatgcatgaggctcttcacaatcactacacacagaagagtctctccttgtctccgggtaaatga

SEQ ID NO: 3 - protein, 469 aa, synthetic construct
  REGION 1..469: note = SYNTHESIZED
MGWSCIILFLVATATGVHSQVQLQQPGAALVKPGASAMMSCKASGYTFTSYWITWVKQRPGQGLEWIGDIYPGTGRTIYKEKFKTKATLTVDTSSSTAFMQLNSLTSEDSAVYYCARGYGSPYYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 720 bp, synthetic construct
  misc_feature 1..720: note = SYNTHESIZED
  source 1..720: mol_type = other DNA
atgggatggtcttgtatcatccttttcttggttgcaacagctactggtgttcattctgatatcgttttaacacaatctccagcatctttggctgtttctcaaggacaaagggctactatcagttgcaaggcttctcaatcagtggactatgacggtgattcttatgttaactggtaccaacagaaacctggacagtccccaaaacttcttatctacgacgcatctaatctcgaatctggaataccagccagattttctggatctggatctggaacagatttcactcttaacatccatcctgtcgaggaagaggatgttgcaacttattactgtcaggaatcaaatgaagatccacgtactttcggtggaggaactaagttggagatcaaagctagcagaactgttgctgcaccatctgttttcatcttccctccatctgatgagcagttgaaatctggaactgcttctgttgtgtgccttcttaataacttctatcctagagaggctaaagttcagtggaaggtggataacgcacttcaatctggtaactctcaagagtctgttacagagcaagattctaaggactcaacttactctctttcatctacacttactttgtcaaaagcagattacgagaaacacaaagtttacgcatgcgaagttactcatcaaggactttcttcaccagttacaaagtctttcaatagaggagagtgttaa

SEQ ID NO: 5 - protein, 239 aa, synthetic construct
  REGION 1..239: note = SYNTHESIZED
MGWSCIILFLVATATGVHSDIVLTQSPASLAVSQGQRATISCKASQSVDYDGDSYVNWYQQKPGQSPKLLIYDASNLESGIPARFSGSGSGTDFTLNIHPVEEEDVATYYCQESNEDPRTFGGGTKLEIKASRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - DNA, 1407 bp, synthetic construct
  misc_feature 1..1407: note = SYNTHESIZED
  source 1..1407: mol_type = other DNA
atgggatggtcttgtatcatccttttcttggttgcaacagctactggtgttcattctgaagttaggttggtggaatcaggaggcggtcttgagcagccagggggttcactgaagctatcttgtgcagcttctggttttacattcagtgattactttatgtattgggtgagacaaacccctgagaaacgtcttgagtgggtcgcctacattagtaatgggggaatttccactttctatagcgatgcagtaaaaggaagattcactatatccagagacaacgctagaaatactctttatctccaaatgtctagattgaagtcagaagatactgctatctactattgtgttaggcaggtatatggccagggatactttgactattggggacaaggtactactttagcagtttcttctgctagcaccaagggaccttctgtttttccacttgctccttcttctaagtctacttctggtggaactgctgctttgggttgtttggtgaaagattactttcctgagccagtgaccgtttcttggaactcaggtgctcttacatctggtgttcatactttcccagctgttcttcaatcttcaggactttactcactttcttctgttgttaccgttccttcttcaagcttgggcactcagacctacatctgcaatgtgaatcacaaacccagcaacaccaaggttgacaagaaagttgagcccaagtcttgtgacaagactcatacgtgtccaccgtgcccagcacctgaacttcttggaggaccgtcagtcttcttgtttcctccaaagcctaaggataccttgatgatctccaggactcctgaagtcacatgtgtagttgtggatgtgagccatgaagatcctgaggtgaagttcaactggtatgtggatggtgtggaagtgcacaatgccaagacaaagccgagagaggaacagtacaacagcacgtacagggttgtctcagttctcactgttctccatcaagattggttgaatggcaaagagtacaagtgcaaggtgtccaacaaagccctcccagcccccattgagaagaccatttccaaagcgaaagggcaaccccgtgaaccacaagtgtacacacttcctccatctcgcgatgaactgaccaagaaccaggtcagcttgacttgcctggtgaaaggcttctatccctctgacatagctgtagagtgggagagcaatgggcaaccggagaacaactacaagactacacctcccgttctcgattctgacggctccttcttcctctacagcaagctcacagtggacaagagcaggtggcaacaagggaatgtcttctcatgctccgtgatgcatgaggctcttcacaatcactacacacagaagagtctctccttgtctccgggtaaatga

SEQ ID NO: 7 - protein, 468 aa, synthetic construct
  REGION 1..468: note = SYNTHESIZED
MGWSCIILFLVATATGVHSEVRLVESGGGLEQPGGSLKLSCAASGFTFSDYFMYWVRQTPEKRLEWVAYISNGGISTFYSDAVKGRFTISRDNARNTLYLQMSRLKSEDTAIYYCVRQVYGQGYFDYWGQGTTLAVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - DNA, 714 bp, synthetic construct
  misc_feature 1..714: note = SYNTHESIZED
  source 1..714: mol_type = other DNA
atgggatggtcttgtatcatccttttcttggttgcaacagctactggtgttcattctcaaatcgttctgattcaatctccagcaattatgtctgcttctcttggagagagggtgactatgacttgcaccgctagtagctctgtttcaagttcttatttgcactggtaccaacagaaacctggatcatcaccaaaactttggatctactcctcattttctttagcatctggagttccagccagattttctggatctggatctggaacatcctatagccttactatctcaacaatggaagctgaggatgctgcaacatattactgtcatcagtatctccgttccccatggactttcggtggaggaagtaagttggagatcaaagctagcagaactgttgctgcaccatctgttttcatcttccctccatctgatgagcagttgaaatctggaactgcttctgttgtgtgccttcttaataacttctatcctagagaggctaaagttcagtggaaggtggataacgcacttcaatctggtaactctcaagagtctgttacagagcaagattctaaggactcaacttactctctttcatctacacttactttgtcaaaagcagattacgagaaacacaaagtttacgcatgcgaagttactcatcaaggactttcttcaccagttacaaagtctttcaatagaggagagtgttaatga

SEQ ID NO: 9 - protein, 236 aa, synthetic construct
  REGION 1..236: note = SYNTHESIZED
MGWSCIILFLVATATGVHSQIVLIQSPAIMSASLGERVTMTCTASSSVSSSYLHWYQQKPGSSPKLWIYSSFSLASGVPARFSGSGSGTSYSLTISTMEAEDAATYYCHQYLRSPWTFGGGSKLEIKASRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - DNA, 432 bp, synthetic construct
  misc_feature 1..432: note = SYNTHESIZED
  source 1..432: mol_type = other DNA
gaattcacaatgggatggtcttgtatcatccttttcttggttgcaacagctactggtgttcattctcaagttcaattgcagcagcctggtgcagctctcgtgaagccaggtgcttcagcaatgatgtcttgtaaggcttctggttacacattcacaagctattggattacatgggtgaaacaaagacctggtcagggtcttgagtggataggagatatttatcccggaactggaagaactatctacaaagagaagtttaaaaccaaggctactctgactgtagataccagttcttctactgcttttatgcaacttaattctttgacatcagaagatagtgctgtgtactattgtgctagggggtatggctcaccatattacgctttagactattggggacaaggtacttccgttactgtgtcttctgctagc

SEQ ID NO: 11 - protein, 141 aa, synthetic construct
  REGION 1..141: note = SYNTHESIZED
MGWSCIILFLVATATGVHSQVQLQQPGAALVKPGASAMMSCKASGYTFTSYWITWVKQRPGQGLEWIGDIYPGTGRTIYKEKFKTKATLTVDTSSSTAFMQLNSLTSEDSAVYYCARGYGSPYYALDYWGQGTSVTVSSAS

SEQ ID NO: 12 - DNA, 399 bp, synthetic construct
  misc_feature 1..399: note = SYNTHESIZED
  source 1..399: mol_type = other DNA
gaattcacaatgggatggtcttgtatcatccttttcttggttgcaacagctactggtgttcattctgatatcgttttaacacaatctccagcatctttggctgtttctcaaggacaaagggctactatcagttgcaaggcttctcaatcagtggactatgacggtgattcttatgttaactggtaccaacagaaacctggacagtccccaaaacttcttatctacgacgcatctaatctcgaatctggaataccagccagattttctggatctggatctggaacagatttcactcttaacatccatcctgtcgaggaagaggatgttgcaacttattactgtcaggaatcaaatgaagatccacgtactttcggtggaggaactaagttggagatcaaa

SEQ ID NO: 13 - protein, 130 aa, synthetic construct
  REGION 1..130: note = SYNTHESIZED
MGWSCIILFLVATATGVHSDIVLTQSPASLAVSQGQRATISCKASQSVDYDGDSYVNWYQQKPGQSPKLLIYDASNLESGIPARFSGSGSGTDFTLNIHPVEEEDVATYYCQESNEDPRTFGGGTKLEIK

SEQ ID NO: 14 - DNA, 429 bp, synthetic construct
  misc_feature 1..429: note = SYNTHESIZED
  source 1..429: mol_type = other DNA
gaattcacaatgggatggtcttgtatcatccttttcttggttgcaacagctactggtgttcattctgaagttaggttggtggaatcaggaggcggtcttgagcagccagggggttcactgaagctatcttgtgcagcttctggttttacattcagtgattactttatgtattgggtgagacaaacccctgagaaacgtcttgagtgggtcgcctacattagtaatgggggaatttccactttctatagcgatgcagtaaaaggaagattcactatatccagagacaacgctagaaatactctttatctccaaatgtctagattgaagtcagaagatactgctatctactattgtgttaggcaggtatatggccagggatactttgactattggggacaaggtactactttagcagtttcttctgctagc

SEQ ID NO: 15 - protein, 140 aa, synthetic construct
  REGION 1..140: note = SYNTHESIZED
MGWSCIILFLVATATGVHSEVRLVESGGGLEQPGGSLKLSCAASGFTFSDYFMYWVRQTPEKRLEWVAYISNGGISTFYSDAVKGRFTISRDNARNTLYLQMSRLKSEDTAIYYCVRQVYGQGYFDYWGQGTTLAVSSAS

SEQ ID NO: 16 - DNA, 390 bp, synthetic construct
  misc_feature 1..390: note = SYNTHESIZED
  source 1..390: mol_type = other DNA
gaattcacaatgggatggtcttgtatcatccttttcttggttgcaacagctactggtgttcattctcaaatcgttctgattcaatctccagcaattatgtctgcttctcttggagagagggtgactatgacttgcaccgctagtagctctgtttcaagttcttatttgcactggtaccaacagaaacctggatcatcaccaaaactttggatctactcctcattttctttagcatctggagttccagccagattttctggatctggatctggaacatcctatagccttactatctcaacaatggaagctgaggatgctgcaacatattactgtcatcagtatctccgttccccatggactttcggtggaggaagtaagttggagatcaaa

SEQ ID NO: 17 - protein, 127 aa, synthetic construct
  REGION 1..127: note = SYNTHESIZED
MGWSCIILFLVATATGVHSQIVLIQSPAIMSASLGERVTMTCTASSSVSSSYLHWYQQKPGSSPKLWIYSSFSLASGVPARFSGSGSGTSYSLTISTMEAEDAATYYCHQYLRSPWTFGGGSKLEIK